(19) United States
(12) Patent Application Publication
(10) Pub. No.: US 2006/0263781 A1
(43) Pub. Date: Nov. 23, 2006

(54) Regulation of cell surface proteins

(76) Inventors: Jacqueline Dalby-Payne, New South Wales (AU); Edward O'Loughlin, New South Wales (AU); Peter Gunning, New South Wales (AU)

Correspondence Address: LICATA & TYRRELL P.C., 66 E. MAIN STREET, MARLTON, NJ, 08053, US

(21) Appl. No.: 10/549,301

(22) PCT Filed: Mar. 22, 2004
(86) PCT No.: PCT/AU04/00358

§ 371 Date: Jul. 17, 2006

(30) Foreign Application Priority Data
Mar. 21, 2003 (AU) 20033901316

(51) Int. Cl. C12Q 1/68; G01N 33/53

(52) U.S. Cl. 435/6; 435/7.1

(57) ABSTRACT

The invention relates to methods of screening for compounds that regulate the activity of cell surface proteins. The present invention relates to a method for regulating the insertion or retention of a protein, such as the cystic fibrosis transmembrane conductance regulator (CFTR), in a cell surface membrane. The invention also relates to methods for the diagnosis and treatment or prevention of diseases caused by abnormal insertion or activity of a cell surface membrane protein, such as cystic fibrosis.

### FIELD OF THE INVENTION

The present invention relates to a method for regulating the insertion or retention of a protein in a cell surface membrane. The invention also relates to methods for the diagnosis and treatment or prevention of diseases caused by abnormal insertion or activity of a cell surface membrane protein, such as cystic fibrosis. The invention further relates to methods of screening for compounds that regulate the transport of molecules into or out of a cell, and for compounds that regulate the activity of cell surface proteins.

### BACKGROUND OF THE INVENTION

The establishment and maintenance of cell polarity is intrinsic to the function of an epithelial cell. The creation of these distinct functional domains relates to their role in providing a barrier and controlling ion and solute transport. Events leading to the development of this functional polarisation include cell-cell contact mediated by E-cadherin and cell-extracellular matrix adherence mediated by integrins. These spatial cues are communicated to the internal components of the cell via localised assembly of cytoskeletal and signalling complexes. This in turn directs reorganisation of the cell surface and the secretory system. The actin cytoskeleton, by virtue of its direct interaction with both integrin- and cadherin-containing complexes, plays a pivotal role in the establishment of epithelial cell polarity. Similarly, the actin filament system is responsible for targeting secretion in budding yeast. Thus, the actin cytoskeleton appears to play a role in the establishment of polarity in different phylla.

Polarised function of the actin cytoskeleton may go beyond specific interactions of actin filaments with integrin and cadherin containing complexes. There is increasing evidence that the isoform composition of actin filaments themselves can differ at different sites in a cell. In gastric parietal cells, the β and γ actin isoforms are differentially distributed in the cell with β actin located predominantly at the more metabolically active apical surface. Similar polarisation of β and γ actin is observed in adult neurons. Polarisation also extends to mRNA location where β, but not γ actin mRNA is specifically located at peripheral sites in the cell associated with motility such as lamellapodia and growth cones.

There are a number of disease states in which alterations in epithelial cell polarity or defects in the process of targeted protein delivery are important features. In autosomal polycystic kidney disease, for example, aberrant expression of basolateral proteins on the apical membrane results in the production of fluid filled cysts. It has recently been reported that 3 cytoskeletal binding proteins required for basolateral membrane organisation, E-cadherin, sec6 and sec8, are abnormally located within the diseased cells. This results in impaired delivery of proteins and lipids to the basolateral membrane (Charron et al., 2000, Journal of Cell Biology 149, 111-124).

Cystic fibrosis is an autosomal recessive condition commonly due to the mutation ΔF508. This mutation results in abnormalities of the cystic fibrosis transmembrane conductance regulator (CFTR) chloride channel. In cystic fibrosis due to the ΔF508 mutation, the CFTR is abnormally folded and so is retained and degraded by the RER (Qu et al., 1997, Journal of Bioenergetics & Biomembranes 29, 483-490; Brown and Breton, 2000, Kidney International 57, 816-824). In addition, CFTR with the ΔF508 mutation has a shorter half-life in the apical membrane (Heda et al., 2001, American Journal of Physiology—Cell Physiology 280, C166-C174).

In cystic fibrosis patients, there is a reduced amount of the CFTR protein in the cell surface of the lung epithelia. It has been shown in the past that the common mutant version of CFTR called deltaF508 gets caught in the interior of the cell and very few copies of it ever make it to the surface of the cell where it belongs. One theory has been that the CFTR protein gets caught simply because it does not fold fast enough, and these mis-folded proteins are degraded before they have a chance to get to their destinations (i.e. the surface of the cell). It is also possible that CFTR is caught in the cell because it is bound by another protein inside the cell, for example BAP31. Any agent that can increase the availability of CFTR to the surface of the cell is therefore a potential therapeutic for the treatment of cystic fibrosis.

Alterations in the distribution of cytoskeletal proteins have also been observed in renal proximal tubule cells in response to ischaemia (Brown et al., 1997, American Journal of Physiology 273, F1003-F1012). In rat kidneys, one hour of ischaemia and reperfusion was found to result in the relocation of the brush border proteins, villin and actin to the basolateral pole (Brown et al., 1997, American Journal of Physiology 273, F1003-F1012). Partial restoration was seen twenty-four hours after reperfusion with full recovery occurring within five days. The authors postulated that the disassembly of the cortical actin cytoskeleton might allow changes in cell shape allowing surviving cells to cover areas of cell loss. In another study, it was found that ischaemia of renal proximal tubule cells resulted in dissociation of tropomyosin from F actin with the tropomyosin relocating to the distal aspects of the microvilli. These authors suggested that the relocation of tropomyosin allows a competing actin binding protein, actin depolymerising factor (ADF), to disrupt the apical microfilaments and thus apical microvilli.

Methods for the diagnosis and treatment of disease states caused by alterations in epithelial cell polarity or defects in the process of targeted protein delivery (such as cystic fibrosis) are highly desirable.

### SUMMARY OF THE INVENTION

The present inventors have investigated the composition of actin microfilaments in gastrointestinal epithelial cells and their role in the delivery of the cystic fibrosis transmembrane conductance regulator (CFTR) into the apical membrane. This investigation has revealed a specific population of microfilaments containing tropomyosin isoforms that are polarised in cell monolayers. Polarisation of this microfilament population occurs very rapidly in response to cell-cell and cell-substratum contact and involves the movement of intact microfilaments. Colocalisation of the tropomyosin isoforms and CFTR was observed in long-term cultures. A reduction in expression of the tropomyosin isoforms resulted in an increase in both CFTR surface expression and chloride efflux in response to cAMP stimulation. The results show that tropomyosin isoforms mark an apical population of microfilaments that can regulate the insertion and/or retention of proteins into the plasma membrane.

Accordingly, in a first aspect the present invention provides a method of screening for a compound that regulates the activity of a cell surface protein, the method comprising determining the activity or cellular location of tropomyosin in the presence of a candidate compound, wherein altered tropomyosin activity or cellular location in the presence of the compound when compared to the absence of the compound indicates that the compound regulates the activity of the cell surface protein.

In a preferred embodiment of this aspect, altered cellular location of tropomyosin, preferably loss of polarised distribution, in the presence of the compound indicates that the compound increases the activity of the cell surface protein.

In a further aspect the present invention provides a method of screening for a compound that regulates the transport of molecules into or out of a cell, the method comprising determining the activity or cellular location of tropomyosin in the presence of a candidate compound, wherein altered tropomyosin activity or cellular location in the presence of the compound when compared to the absence of the compound indicates that the compound regulates the transport of molecules into or out of a cell.

In a preferred embodiment of this aspect, altered cellular location of tropomyosin, preferably loss of polarised distribution, in the presence of the compound indicates that the compound increases the transport of molecules into and/or out of a cell.

In yet a further aspect the present invention provides a method of screening for a therapeutic compound for the treatment of cystic fibrosis, the method comprising determining the activity or cellular location of tropomyosin in the presence of a candidate compound, wherein altered tropomyosin activity or cellular location in the presence of the compound when compared to the absence of the compound indicates that the compound is useful in the treatment of cystic fibrosis.

In a preferred embodiment of this aspect, altered cellular location of tropomyosin, preferably loss of polarised distribution, in the presence of the compound indicates that the compound is useful in the treatment of cystic fibrosis.

In one particular embodiment of these aspects, cellular location of tropomyosin is assessed as an indicator of the ability of the compound to regulate the transport of molecules into or out of a cell or to regulate the activity of a cell surface protein. Cells which normally exhibit polarised distribution of tropomyosin (for example, gastrointestinal epithelial cells, fibroblasts or neurons) are preferably selected for this method of screening. Following exposure of the candidate compound to the selected cells, the location or distribution of tropomyosin is assessed and compared to the location or distribution of tropomyosin in cells that have not been exposed to the candidate compound. In a preferred embodiment, loss of polarised distribution of the tropomyosin in cells that have been exposed to the candidate compound indicates that the candidate compound is capable of increasing the activity of a cell surface protein or that the candidate compound is capable of increasing the transport of molecules into and/or out of a cell, or that the compound is useful in the treatment of cystic fibrosis.

In yet a further aspect the present invention provides a method of screening for a compound that regulates the activity of a cell surface protein, the method comprising determining the expression levels of tropomyosin in the presence of a candidate compound, wherein altered tropomyosin expression in the presence of the compound when compared to the absence of the compound indicates that the compound regulates the activity of the cell surface protein.

In a preferred embodiment of this aspect reduced tropomyosin expression in the presence of the compound indicates that the compound increases the activity of the cell surface protein.

In yet a further aspect the present invention provides a method of screening for a compound that regulates the transport of molecules into or out of a cell, the method comprising determining the expression levels of tropomyosin in the presence of a candidate compound, wherein altered tropomyosin expression in the presence of the compound when compared to the absence of the compound indicates that the compound regulates the transport of molecules into or out of a cell.

In a preferred embodiment of this aspect reduced tropomyosin expression in the presence of the compound indicates that the compound increases the transport of molecules into or out of a cell.

In yet a further aspect the present invention provides a method of screening for a therapeutic compound for the treatment of cystic fibrosis, the method comprising determining the expression levels of tropomyosin in the presence of a candidate compound, wherein altered tropomyosin expression in the presence of the compound when compared to the absence of the compound indicates that the compound is useful in the treatment of cystic fibrosis.

In a preferred embodiment of this aspect reduced tropomyosin expression in the presence of the compound indicates that the compound is useful in the treatment of cystic fibrosis.

In a preferred embodiment, determining the expression level of tropomyosin comprises measuring the amount of the tropomyosin protein and/or mRNA. In one preferred embodiment, the amount of tropomyosin protein is measured using an anti-tropomyosin antibody. In another embodiment, the amount of the tropomyosin-associated transcript (e.g. mRNA) is measured by contacting the sample with a polynucleotide that selectively hybridizes to the tropomyosin transcript.

In yet a further aspect the present invention provides a method of screening for a compound that regulates the activity of a cell surface protein, the method comprising measuring the binding of tropomyosin to one of its binding partners in the presence of a candidate compound, wherein an altered level of binding of tropomyosin to its binding partner in the presence of the compound when compared to the absence of the compound indicates that the compound regulates the activity of a cell surface protein.

In a preferred embodiment of this aspect a reduced level of binding of tropomyosin to its binding partner in the presence of the compound indicates that the compound increases the activity of a cell surface protein.

In yet a further aspect the present invention provides a method of screening for a compound that regulates the transport of molecules into or out of a cell, the method comprising measuring the binding of tropomyosin to one of its binding partners in the presence of a candidate compound, wherein an altered level of binding of tropomyosin to its binding partner in the presence of the compound when compared to the absence of the compound indicates that the compound regulates the transport of molecules into or out of a cell.

In a preferred embodiment of this aspect a reduced level of binding of tropomyosin to its binding partner in the presence of the compound indicates that the compound increases the transport of molecules into or out of a cell.

In yet a further aspect the present invention provides a method of screening for a therapeutic compound for the treatment of cystic fibrosis, the method comprising measuring the binding of tropomyosin to one of its binding partners in the presence of a candidate compound, wherein an altered level of binding of tropomyosin to its binding partner in the presence of the compound when compared to the absence of the compound indicates that the compound is useful in the treatment of cystic fibrosis.

In a preferred embodiment of this aspect a reduced level of binding of tropomyosin to its binding partner in the presence of the compound indicates that the compound is useful in the treatment of cystic fibrosis.

In a further preferred embodiment of these aspects of the invention the tropomyosin binding partner is selected from the group consisting of calponin, CEACAM1, endostatin, Enigma, Gelsolin (preferably sub-domain 2), S100A2 and actin. In a further preferred embodiment, the tropomyosin binding partner is actin.

As will be readily understood by those skilled in this field the methods of the present invention provide a rational method for designing and selecting compounds which interact with and modulate the activity of tropomyosin. In the majority of cases these compounds will require further development in order to increase activity. It is intended that in particular embodiments the methods of the present invention include such further developmental steps. For example, it is intended that embodiments of the present invention further include manufacturing steps such as incorporating the compound into a pharmaceutical composition in the manufacture of a medicament.

Accordingly, in a further aspect, the method further comprises formulating the identified compound for administration to a human or a non-human animal as described herein.

In a further aspect the present invention provides a method for regulating the insertion or retention of a protein in a cell surface membrane, the method comprising administering to the cell an agent that modulates tropomyosin expression, location or activity.

In a further aspect the present invention provides a method for increasing the insertion or retention of a protein in the surface membrane of a cell, the method comprising administering to the cell a tropomyosin antagonist.

In yet a further aspect the present invention provides a method for regulating the transport of molecules into or out of a cell, the method comprising administering to the cell an agent that modulates tropomyosin expression, location or activity.

In yet a further aspect the present invention provides a method for increasing the transport of molecules into or out of a cell, the method comprising administering to the cell a tropomyosin antagonist.

In one embodiment of the invention the molecules transported are selected from the group consisting of electrolytes, water, monosaccharides and ions.

In yet a further aspect the present invention provides a method for the treatment or prevention of a disease in a subject caused by the abnormal insertion, retention or activity of a cell surface membrane protein, the method comprising administering to the subject an agent that modulates tropomyosin expression, location or activity. Preferably, the agent that modulates tropomyosin expression, location or activity is a tropomyosin antagonist.

In a preferred embodiment of the present invention, the cell surface membrane protein is selected from the group consisting of a transport protein, a channel, a receptor, a growth factor, an antigen, a signalling protein and a cell adhesion protein. The transport protein is preferably the cystic fibrosis transmembrane conductance regulator (CFTR).

In a further preferred embodiment of the present invention, the cell is a non-muscle cell. In one preferred embodiment, the cell is a neuronal cell or an epithelial cell. Preferably, the epithelial cell is a gastrointestinal epithelial cell.

The disease caused by the abnormal insertion or activity of a cell surface membrane protein may be, for example, cystic fibrosis, multiple sclerosis, polycistic kidney disease, viral infection, bacterial infection, reperfusion injury, Menkes Disease, Wilson's Disease, diabetes, myotonic dystrophies, epilepsy or mood disorders such as depression, bipolar disorder or dysthymic disorder.

In yet a further aspect the present invention provides a method for the treatment or prevention of a cystic fibrosis in a subject, the method comprising administering to the subject an agent that modulates tropomyosin expression, location or activity. Preferably, the agent that modulates tropomyosin expression, location or activity is a tropomyosin antagonist.

In the context of the present invention, it is preferred that the tropomyosin is an isoform encoded by a human gene selected from the non-limiting group consisting of TPM 1, TPM 2, TPM 3 and TPM 4. For example, the isoform may be selected from the group consisting of TM1, TM2, TM3, TM4, TM5, TM5a, TM5b, TM6, Tm5NM-1, Tm5NM-2, Tm5NM-3, Tm5NM-4, Tm5NM-5, Tm5NM-6, Tm5NM-7, Tm5NM-8, Tm5NM-9, Tm5NM-10, and Tm5NM-11.

In a preferred embodiment, the tropomyosin isoform comprises an amino acid sequence encoded by exon 1b of the TPM 1 gene (SEQ ID NO:11) or an amino acid sequence encoded by exon 1b of the TPM 3 gene (SEQ ID NO:12).

In a further preferred embodiment, the tropomyosin isoform is TM5a (preferably with a sequence as shown in SEQ ID NO:9) or TM5b (preferably with a sequence as shown in SEQ ID NO:10).

A tropomyosin antagonist for use in the present invention may be selected from the group consisting of a peptide, an antibody directed against tropomyosin, a small organic molecule, an antisense compound directed against tropomyosin-encoding mRNA, an anti-tropomyosin catalytic molecule such as a ribozyme or a DNAzyme, and a dsRNA or small interfering RNA (RNAi) molecule that targets tropomyosin expression.

In one preferred embodiment the tropomyosin antagonist is an antisense compound, a catalytic molecule or an RNAi molecule directed against tropomyosin-encoding mRNA. In a further preferred embodiment, the tropomyosin antagonist is an antisense compound, a catalytic molecule or an RNAi molecule targeted specifically against exon 1b of the TPM 1 gene (SEQ ID NO:7) or exon 1b of the TPM 3 gene (SEQ ID NO:8).

In a further preferred embodiment the tropomyosin antagonist is an antisense compound, a catalytic molecule or an RNAi molecule targeted to the sequence AGCTCGCTGGAGGCGGTG (SEQ ID NO:13).

In one particularly preferred embodiment, the tropomyosin antagonist is an antisense compound comprising the sequence CACCGCCUCCAGCGAGCT (SEQ ID NO:14).

In a preferred embodiment the tropomyosin antagonist specifically alters the cellular location of TM5a or TM5b. By “specifically alters the cellular location of TM5a or TM5b” we mean that the compound significantly alters the cellular location of TM5a or TM5b without significantly altering the cellular location of other tropomyosin isoforms.

In another preferred embodiment the tropomyosin antagonist specifically reduces or inhibits TM5a or TM5b expression. By “specifically reduces or inhibits TM5a or TM5b expression” we mean that the compound significantly reduces or inhibits TM5a or TM5b expression without significantly reducing or inhibiting the expression of other tropomyosin isoforms.

In another preferred embodiment the tropomyosin antagonist specifically alters the binding of TM5a or TM5b to one of its binding partners. By “specifically alters the binding of TM5a or TM5b to one of its binding partners” we mean that the compound significantly alters the binding of TM5a or TM5b to one of its binding partners without significantly altering the binding of other tropomyosin isoforms to their binding partners.

In yet a further aspect the present invention provides a method for assessing an individual's predisposition to a disease caused by the abnormal insertion, retention or activity of a cell surface membrane protein, the method comprising the step of determining the presence of a mutation in a tropomyosin gene of the individual.

The mutation in the tropomyosin gene may be a point mutation (i.e. a single nucleotide polymorphism (SNP)), deletion and/or insertion. Such a mutation may be detected by isolating and sequencing DNA fragments from the tropomyosin gene or otherwise by isolating mRNA from the individual and synthesising DNA therefrom (e.g. by RT-PCR) for sequencing. Mutations may also be detected by hybridisation using discriminating oligonucleotide probes or by amplification procedures using discriminating oligonucleotide primers.

In yet a further aspect the present invention provides a method for assessing an individual's predisposition to a disease caused by the abnormal insertion, retention or activity of a cell surface membrane protein, the method comprising analysing the polarised distribution of tropomyosin in the cells of the individual.

If the distribution of a particular tropomyosin isoform differs in cells derived from the individual being tested from that observed in cells of a normal subject, this is indicative that the individual being tested has a predisposition to a disease caused by the abnormal insertion, retention or activity of a cell surface membrane protein.

The present invention also provides kits comprising polynucleotide probes and/or monoclonal antibodies, and optionally quantitative standards, for carrying out methods of the invention. Furthermore, the invention provides methods for evaluating the efficacy of drugs, and monitoring the progress of patients, involved in clinical trials for the treatment of disorders as recited herein.

As will be apparent, preferred features and characteristics of one aspect of the invention are applicable to other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

FIG. 1. Maps of the four tropomyosin (Tm) genes and their product(s). Exons are shown as shaded boxes, the 3′ untranslated sequence as unshaded boxes and the introns are represented by lines. (A) The fast gene (α-Tmf). Note that exon 1b is unique to Tm5a and Tm5b. (B) Tm5NM gene. (C) The β-TM gene. (D) The TM-4 gene (Taken from Temm-Grove C J et al 1998 and Percival et al 2000)

FIG. 2. Tropomyosin antibody specificity. Tropomyosin antibody specificity in T84 cells and human fibroblasts are shown in Western blots. The specificities of 311 in T84 cells (left) and fibroblasts (right) are shown in A and the specificities of αf9d and CG3 antibodies are shown in B and C respectively. The 311 antibody detects Tm6 (40 kDa), Tm2 (36 kDa) and Tm3 (34 kDa) in human fibroblasts. Tm2 is absent in T84 cells. Tm1 (36 kDa) was absent from both T84 cells and human fibroblasts. αf9d detects Tm6 (40 kDa), Tm3 (34 kDa), Tm5a (30 kDa) and Tm5b (30 kDa). CG3 antibody detects 11 possible isoforms that co-migrate at 30 kDa.

FIG. 3. T84 cell monolayers express a polarised distribution of Tm5a and Tm5b. (A-F) Mature T84 cell monolayers were labelled with αf9d (A and B), 311 (C and D) and CG3 antibodies. The antibody distribution was analysed by Confocal Laser Scanning Microscopy. Images in the vertical plane (xz) are shown on the left and images in the horizontal plane (xy) are shown on the right. The differential staining pattern between αf9d and 311 represents Tm5a and Tm5b. Bar, 10 μm. (G) The mean apical and central pixel intensity was measured across the apical and central region of the individual monolayers. The apical: central mean pixel intensity ratios for αf9d and 311 were compared in co-stained monolayers and are represented as the mean±standard deviation for each group. Results represent the average of 8 co-stained monolayers.

FIG. 4. Localisation of tropomyosin isoforms in the crypts and villi of the rat duodenum. Sections of rat duodenal tissue were fixed and stained with αf9d (C and D), 311 (E and F) and CG3 (G and H) antibodies. Sections through the crypts are on the left and sections through the villi are on the right. A and B represent antibody negative controls. Arrows indicate gastrointestinal epithelial cells. Immunoreactivity is indicated by the blue staining and slides were counterstained with Nuclear fast red. Bar, 10 μm.

FIG. 5. The development of polarisation of tropomyosin isoforms. (A-L) Immunofluorescent confocal microscopy images of T84 cells stained for tropomyosin isoforms at various time points after seeding. All images are in the vertical (xz) plane. At each time point, the images on the left and in the centre are of the same co-stained cells. On the left the 311 antibody (Tm 3, 6) staining is shown. In the centre the αf9d antibody (Tm 3, 5a, 5b, 6) staining is shown. The cells on the right are stained with CG3 antibody (TmNM1-11). (A, B and C) 10 minutes; (D, E and F) 1 hour; (G, H and I) 2 hours; (J, K and L) 24 hours. The arrow indicates a T84 cell in suspension with circumferential staining. Bar, 10 μm. (M and N) Total protein and specific tropomyosin isoform expression during T84 cell monolayer development. Protein was extracted from T84 cells 1,2,4 and 24 hours and 7 days after seeding. (M) Gel stained with Coomassie blue showing total protein. (N) Western blot immunoblotted with αf9d antibody (Tm 3, 5a, 5b, 6).

FIG. 6. Localisation of tropomyosin isoforms in T84 cells after treatment with jasplakinolide or nocodazole. Immunofluorescent confocal microscopy images of T84 cells stained for tropomyosin isoforms 10 minutes after cell seeding (A-D) and mature T84 cell monolayer (E and F). All images are in the vertical (xz) plane. Cells on the left are stained with 311 antibody (Tm 3,6) and cells on the right are stained with αf9d antibody (Tm 3, 5a, 5b, 6). (A and B) Cells treated with jasplaldnolide 1 μM. (C and D) Cells treated with nocodazole 33 μM. (E and F) T84 cell monolayers treated with 20 μM cytochalasin D for 3 hours. The arrows indicate a T84 cells in suspension with circumferential staining. Bar, 10 μm.

FIG. 7. T84 cell monolayers co-stained for tropomyosin isoforms and CFTR. Immunofluorescent confocal microscopy images of T84 cell monolayers co-stained for tropomyosin isoforms and CFTR. All images are in the vertical plane. (A) αf9d antibody (Tm 3,5a,5b,6). The arrow indicates area of enriched αf9d staining in the apical membrane not associated with CFTR; (B) CFTR antibody. The arrow indicates CFTR located in the cytoplasm; (C) Overlay of image A and image B. Bar, 10 μm.

FIG. 8. Effect of antisense and nonsense oligonucleotides against Tm5a and Tm5b on the distribution of αf9d antibody staining in T84 cell monolayers. Immunofluorescent confocal microscopy images of T84 cell monolayers. Both images are in the vertical plane. Both monolayers have been stained with αf9d (Tm3, 5a, 5b, 6). (A) Nonsense oligonucleotide 2 μM for 24 hours; (B) Antisense oligonucleotide 2 μM for 24 hours. Bar, 10 μm. (C and D) Western blot showing the effect of antisense and nonsense oligonucleotides against Tm5a and Tm5b on T84 cells. Protein was extracted from T84 cell monolayers following treatment with either 2 μM antisense or nonsense oligonucleotides against Tm5a and Tm5b for 24 hours. (C) Gel stained with Coomassie blue showing total protein. (D) Western blot immunoblotted with the αf9d antibody (Tm3, 5a, 5b, 6). (E) Effect of antisense and nonsense oligonucleotides against Tm5a and Tm5b on intensity of apical staining with αf9d antibody in T84 cell monolayers. The apical αf9d antibody staining pixel intensity was determined by confocal microscope in T84 cell monolayers treated with either 2 μM antisense or nonsense oligonucleotides for 24 hours. The mean±1 SD for each group is depicted. (Nonsense 150.86±48.28, Antisense 53.62±31.62; p<0.001)

FIG. 9. Effect of antisense and nonsense oligonucleotides against Tm5a and Tm5b on cell surface expression of CFTR and chloride efflux in T84 cell monolayers. (A) Enzyme linked CFTR surface expression assays were performed on T84 cell monolayers treated with either 2 μM antisense or nonsense oligonucleotides for 24 hours. CFTR expression is represented by absorbance at 655 nm, normalised to the mean absorbance of the nonsense treated group within individual experiments. The mean±1 SD for each group is depicted. (Nonsense 1±0.42, Antisense 1.49±0.78; p<0.001). (B) MQAE chloride efflux assays were performed on control T84 cell monolayers treated with either 2 μM antisense or nonsense oligonucleotides against Tm5a and Tm5b. Cumulative chloride efflux at 15 minutes is represented by the mean percentage increase in fluorescence from baseline, normalised to the mean percentage increase of the nonsense group, within individual experiments. The mean±1 SD for each group is depicted. (Nonsense 1±0.36, Antisense 1.47±0.41; p<0.001)

FIG. 10. Effect of nocodazole treatment on cell surface expression of CFTR and chloride efflux in T84 cell monolayers. Enzyme linked CFTR surface expression assays were performed on forskolin stimulated T84 cell monolayers with and without treatment with 33 μM nocodazole for 3 hours. CFTR expression is represented by absorbance at 655 nm, normalised to the mean absorbance of the control group within individual experiments. The mean±SD for each group is depicted. (Control 1.00±0.29, Nocodazole 0.92±0.25; p=0.64). (B) MQAE chloride efflux assays were performed on control T84 cell monolayers and T84 cell monolayers treated with nocodazole 33 μM for 3 hours. Cumulative chloride efflux at 15 minutes is represented by the mean percentage increase in fluorescence from baseline, normalised to the mean percentage increase of the control group, within individual experiments. The mean±SD for each group is depicted. (Control 1.00±0.22, Nocodazole 1.01±0.43; p=0.93).

### KEY TO SEQUENCE LISTING

SEQ ID NO:1: Homo sapiens cDNA sequence for an isoform encoded by the tropomyosin 1 (alpha) (TPM 1) gene sequence;

SEQ ID NO:2: Homo sapiens cDNA sequence for an isoform encoded by the tropomyosin 2 (beta) (TPM 2) gene sequence;

SEQ ID NO:3: Homo sapiens cDNA sequence for an isoform encoded by the tropomyosin 3 (TPM 3) gene sequence;

SEQ ID NO:4: Homo sapiens cDNA sequence for an isoform encoded by the tropomyosin 4 (TPM 4) gene sequence;

SEQ ID NO:5: Homo sapiens cDNA sequence of isoform TM5a;

SEQ ID NO:6: Homo sapiens cDNA sequence of isoform TM5b;

SEQ ID NO:7: Homo sapiens DNA sequence of exon 1b of the TPM1 gene;

SEQ ID NO:8: Homo sapiens DNA sequence of exon 1b of the TPM3 gene;

SEQ ID NO:9: Homo sapiens protein sequence of isoform TM5a;

SEQ ID NO:10: Homo sapiens protein sequence of isoform TM5b;

SEQ ID NO:11: Homo sapiens protein sequence of exon 1b of the TPM1 gene;

SEQ ID NO:12: Homo sapiens protein sequence of exon 1b of the TPM3 gene;

SEQ ID NO:13: Homo sapiens target sequence within exon 1b of the TPM1 gene for preferred antisense constructs;

SEQ ID NO:14: Antisense oligonucleotide sequence targeted to exon 1b of the TPM1 gene;

SEQ ID NO:15: Nonsense oligonucleotide sequence (control sequence);

SEQ ID NOs:16 and 17: Polynucleotides for producing siRNA molecules which downregulate human TM5a or TM5b production;

SEQ ID NOs:18-20—Antigenic epitopes in the amino acid sequence encoded by exon 1b of the TMP1 gene.

### DETAILED DESCRIPTION OF THE PREFERRED EMBODIMENTS

Unless defined otherwise, all technical and scientific terms used herein have the same meaning as commonly understood by one of ordinary skill in the art (e.g. in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 3rd ed. (2001) Cold Spring Harbor Laboratory Press, Cold Spring Harbor, N.Y. and Ausubel et al., Short Protocols in Molecular Biology (1999) 4th Ed, John Wiley & Sons, Inc.—and the full version entitled Current Protocols in Molecular Biology, which are incorporated herein by reference) and chemical methods.

Tropomyosins

The present invention is based on the finding that insertion, retention or maintenance of proteins in the surface membrane of a cell is regulated by tropomyosin. This finding provides the basis for diagnostic and therapeutic methods relating to diseases that are caused by abnormal insertion or functioning of cell surface proteins.

Tropomyosins (TMs) are a diverse group of proteins found in all eukaryotic cells, with distinct isoforms found in muscle (skeletal, cardiac and smooth), brain and various non-muscle cells. They are elongated proteins that possess a simple dimeric α-helical coiled coil structure along their entire length. The coiled coil structure is based on a repeated pattern of seven amino acids with hydrophobic residues at the first and fourth positions and is highly conserved in all TM isoforms found in eukaryotic organisms from yeast to man with a prominent seven-residue periodicity (five motifs). Different isoforms are produced by differential splicing; e.g isoforms of α-tropomyosin differ in striated and smooth muscle.

TMs are associated with the thin filaments in the sarcomeres of muscle cells and the microfilaments of non-muscle cells. The TMs bind to themselves in a head-to-tail manner, and lie in the groove of F-actin, with each molecule interacting with six or seven actin monomers.

The function of TM in skeletal and cardiac muscle is, in association with the troponin complex (troponins T, C and I), to regulate the calcium-sensitive interaction of actin and myosin. Under resting intracellular calcium ion concentrations, the troponin-tropomyosin complex inhibits actomyosin ATPase activity. When a stimulus induces calcium ion release in the muscle cell, troponin-C binds additional calcium ions and a conformational change is transmitted through the troponin-tropomyosin complex which releases the inhibition of actomyosin ATPase activity, resulting in contraction.

In contrast to the skeletal and cardiac muscle, the biological functions of smooth muscle and non-muscle TMs are poorly understood. Smooth muscle and non-muscle cells are devoid of a troponin complex and the phosphorylation of the light chains of myosins appears to be the major calcium-sensitive regulatory mechanism controlling the interaction of actin and myosin. These differences in the regulation of contractile apparatus of various cell types appear to require structurally as well as functionally distinct forms of TM.

When used herein the term “tropomyosin” is intended to encompass all isoforms of the protein. For example, the term encompasses all isoforms encoded by the mammalian genes TPM 1 (also known as the alpha-TM gene) (MacLeod and Gooding, 1988, Mol. Cell. Biol. 8, 433-440), TPM 2 (also known as the beta-TM gene) (MacLeod et al., 1985, Proc. Natl. Acad. Sci. USA 82, 7835-7839), TPM 3 (also known as the gamma-TM gene) (Clayton et al., 1988, J. Mol. Biol. 201, 507-515), and TPM 4 (also known as the delta-TM gene) (MacLeod et al., 1987, J. Mol. Biol. 194, 1-10).

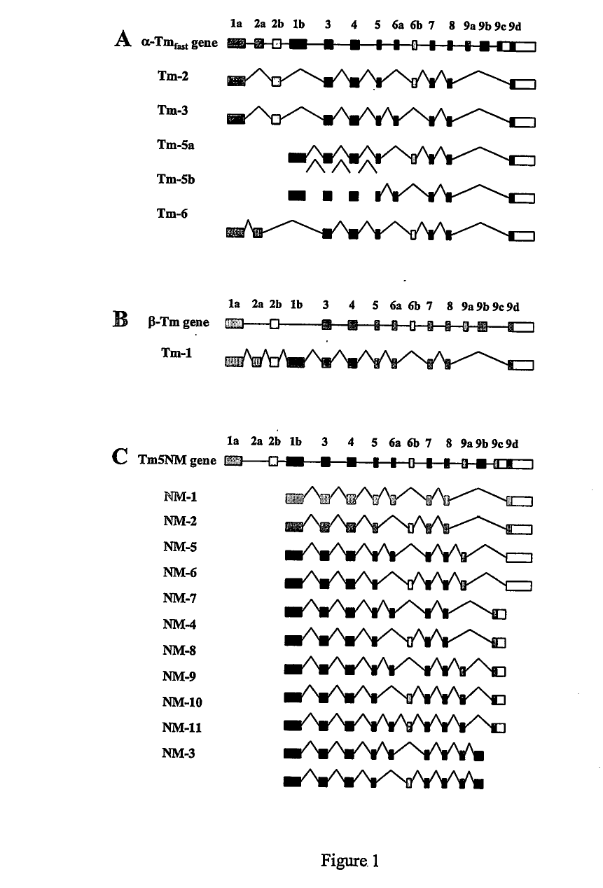

There are at least 40 tropomyosin isoforms that are derived from these four genes by alternative splicing (FIG. 1). See, for example, Lees-Miller and Helfinan, 1991, Bioessays 13(9):429-437. Although tropomyosin isoforms have a high degree of similarity, there are some differences in the actin binding and head-tail binding domains. The various tropomyosm isoforms have different binding affinities for actin and this is thought to result in a differential effect on the stability of actin microfilaments. In addition, the tropomyosin position on the actin microfilament may modulate actin's role in cell motility and cytoskeletal remodelling. Once inserted, tropomyosins influence the interaction between actin and other actin binding proteins. For example, high molecular weight troposmyosins are protective against the severing activity of the actin binding protein gelsolin.

cDNA sequences of isoforms encoded by the human TPM1, TMP2, TPM3 and TPM4 genes are shown in SEQ ID NOs 1 to 4 respectively. These sequences are representative examples only and are not intended to limit the scope of the present invention. The methods of the present invention may be targeted to other human or non-human tropomyosin sequences.

Diagnostic Analysis

In one aspect the present invention relates to a method for predicting the likelihood that an individual has a predisposition to a disease caused by abnormal insertion, retention or function of a cell surface protein, or for aiding in the diagnosis of such as disease.

In one embodiment the diagnostic method comprises the steps of obtaining a polynucleotide sample from an individual to be assessed and analysing a tropomyosin gene.

The genetic material to be assessed can be obtained from any nucleated cell from the individual. For assay of genomic DNA, virtually any biological sample (other than pure red blood cells) is suitable. For example, convenient tissue samples include whole blood, semen, saliva, tears, urine, fecal material, sweat, skin, testis, placenta, kidney and hair. For assay of cDNA or mRNA, the tissue sample is preferably obtained from an organ in which the target nucleic acid is expressed. For example, epithelial cells are suitable sources for obtaining cDNA for tropopmyosin genes.

The analysis of the tropomyosin gene may require amplification of DNA from target samples. This can be accomplished by e.g., PCR. See generally PCR Technology: Principles and Applications for DNA Amplification (ed. H. A. Erlich, Freeman Press, New York, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleotide which occupies a polymorphic site of interest can be identified by a variety methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; exon trapping, single base extension (SBE); or analysis of the tropomyosin protein.

In another embodiment the diagnostic method involves analysing the polarised distribution of tropomyosin in the cells of the individual. This analysis may be conducted, for example, by antibody staining of a particular tropomyosin isoform within cells (preferably epithelial cells) derived from the individual being tested.

Tropomyosin Antagonists/Agonists

In one aspect the present invention relates to methods of screening for compounds that regulate tropomyosin activity or location within a cell.

In certain embodiments, combinatorial libraries of potential modulators will be screened for an ability to bind to a tropomyosin or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., 1994, J. Med. Chem. 37(9):1233-1251).

Preparation and screening of combinatorial chemical libraries is well known to those of skill in the art. Such combinatorial chemical libraries include, but are not limited to, peptide libraries, peptoids, encoded peptides, random bio-oligomers, nonpeptidal peptidomimetics, analogous organic syntheses of small compound libraries, nucleic acid libraries, peptide nucleic acid libraries, antibody libraries, carbohydrate libraries and small organic molecule libraries.

Tropomyosin binding compounds can be readily identified and isolated by methods known to those of skill in the art. Examples of methods that may be used to identify tropomyosin binding compounds are the yeast-2-hybrid screening, phage display, affinity chromatography, expression cloning and Biacore systems. Biacore systems are used to identify chemical mimetics of a tropomyosin protein as these systems enable direct detection and monitoring of biomolecular binding events in real time without labeling and often without purification of the substances involved. (Biacore, Rapsagatan 7, SE 754 50 Uppsala.).

In particular, the yeast-2-hybrid screening approach utilizes transcription activation to detect protein-protein interactions. Many transcription factors can be separated into two domains, a DNA binding domain and a transcriptional activation domain that are inactive when separated. When the two domains are brought into ‘close proximity’ their functional transcriptional activation activity is recreated. In the present invention, a tropomyosin protein is fused to a transcription factor DNA binding domain and cDNAs from a cDNA library are fused to a sequence encoding a transcriptional activation domain. A yeast strain which has been transformed with the cDNA encoding the protein of interest fused to a transcription factor DNA binding domain, is transformed with the transcriptional activation domain/cDNA fusion library. Any cDNA which codes a protein that binds to the protein of interest will allow the formation of a functional hybrid transcriptional activator (as the DNA binding and transcriptional activation domains are now in ‘close proximity’) leading to the expression of a reporter gene that results in cell survival. The cDNA coding the binding protein is then isolated and the protein that it encodes identified. The assays to identify modulators are preferably amenable to high throughput screening. Preferred assays thus detect enhancement or inhibition of tropomyosin gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detectors) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

Protein or Peptide Inhibitors

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, peptidyl tropomyosin inhibitors are chemically or recombinantly synthesized as oligopeptides (approximately 10-25 amino acids in length) derived from the tropomyosin sequence. Alternatively, tropomyosin fragments are produced by digestion of native or recombinantly produced tropomyosin by, for example, using a protease, e.g., trypsin, thermolysin, chymotrypsin, or pepsin. Computer analysis (using commercially available software, e.g. MacVector, Omega, PCGene, Molecular Simulation, Inc.) is used to identify proteolytic cleavage sites. The proteolytic or synthetic fragments can comprise as many amino acid residues as are necessary to partially or completely inhibit tropomyosin function. Preferred fragments will comprise at least 5, 10, 15, 20, 25, 30, 35, 40, 45, 50, 55, 60, 65, 70, 75, 80, 85, 90, 95, 100 or more amino acids in length.

Protein or peptide inhibitors may also be dominant-negative mutants of tropomyosin. The term “dominant-negative mutant” refers to a tropomyosin polypeptide that has been mutated from its natural state and that interacts with a protein that tropomyosin normally interacts with thereby preventing endogenous native tropomyosin from forming the interaction.

Anti-Tropomyosin Antibodies

The term “antibody” as used in this invention includes intact molecules as well as fragments thereof, such as. Fab, F(ab′)2, and Fv which are capable of binding an epitopic determinant of tropomyosin. These antibody fragments retain some ability to selectively bind with its antigen and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

Antibodies of the present invention can be prepared using intact tropomyosin or fragments thereof as the immunizing antigen. A peptide used to immunize an animal can be derived from translated cDNA or chemical synthesis and is purified and conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide may then be used to immunize the animal (e.g., a mouse or a rabbit).

If desired, polyclonal antibodies can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1991, incorporated by reference).

Monoclonal antibodies may be prepared using any technique which provides for the production of antibody molecules by continuous cell lines in culture, such as, for example, the hybridoma technique, the human B-cell hybridoma technique, and the EBV-hybridoma technique (Kohler et al. Nature 256, 495-497, 1975; Kozbor et al., J. Immunol. Methods 81, 31-42, 1985; Cote et al., Proc. Natl. Acad. Sci. USA 80, 2026-2030, 1983; Cole et al., Mol. Cell Biol. 62, 109-120, 1984).

Methods known in the art allow antibodies exhibiting binding for tropomyosin to be identified and isolated from antibody expression libraries. For example, a method for the identification and isolation of an antibody binding domain which exhibits binding to tropomyosin is the bacteriophage lambda vector system. This vector system has been used to express a combinatorial library of Fab fragments from the mouse antibody repertoire in Escherichia coli (Huse, et al., Science, 246:1275-1281, 1989) and from the human antibody repertoire (Mullinax, et al., Proc. Nat. Acad. Sci., 87:8095-8099, 1990). This methodology can also be applied to hybridoma cell lines expressing monoclonal antibodies with binding for a preselected ligand. Hybridomas which secrete a desired monoclonal antibody can be produced in various ways using techniques well understood by those having ordinary skill in the art and will not be repeated here. Details of these techniques are described in such references as Monoclonal Antibodies-Hybridomas: A New Dimension in Biological Analysis, Edited by Roger H. Kennett, et al., Plenum Press, 1980; and U.S. Pat. No. 4,172,124, incorporated by reference.

In addition, methods of producing chimeric antibody molecules with various combinations of “humanized” antibodies are known in the art and include combining murine variable regions with human constant regions (Cabily, et al. Proc. Natl. Acad. Sci. USA, 81:3273, 1984), or by grafting the murine-antibody complementarity determining regions (CDRs) onto the human framework (Riechmann, et al., Nature 332:323, 1988).

In one embodiment, the antibody binds at least a portion of a region of human tropomyosin selected from, but not limited to, the group consisting of SEQ ID NOs: 18-20.

Antisense Compounds

The term “antisense compounds” encompasses DNA or RNA molecules that are complementary to at least a portion of a tropomyosin mRNA molecule (Izant and Weintraub, Cell 36:1007-15, 1984; Izant and Weintraub, Science 229(4711):345-52, 1985) and capable of interfering with a post-transcriptional event such as mRNA translation. Antisense oligomers complementary to at least about 15 contiguous nucleotides of tropomyosin-encoding mRNA are preferred, since they are easily synthesized and are less likely to cause problems than larger molecules when introduced into the target tropomyosin-producing cell. The use of antisense methods is well known in the art (Marcus-Sakura, Anal. Biochem. 172: 289, 1988). Preferred antisense nucleic acid will comprise a nucleotide sequence that is complementary to at least 15 contiguous nucleotides of a sequence encoding the amino acid sequence set forth in SEQ ID NO:7 or SEQ ID NO:8.

Catalytic Nucleic Acids

The term catalytic nucleic acid refers to a DNA molecule or DNA-containing molecule (also known in the art as a “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity (also referred to herein as the “catalytic domain”). To achieve specificity, preferred ribozymes and DNAzymes will comprise a nucleotide sequence that is complementary to at least about 12-15 contiguous nucleotides of a sequence encoding a tropomyosin isoform.

The types of ribozymes that are particularly useful in this invention are the hammerhead ribozyme (Haseloff and Gerlach 1988, Perriman et al., 1992) and the hairpin ribozyme (Shippy et al., 1999).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for the ribozymes of this invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase. Alternatively, the ribozyrne can be modified to the phosphothio analog for use in liposome delivery systems. This modification also renders the ribozyme resistant to endonuclease activity.

RNA Inhibitors

dsRNA is particularly useful for specifically inhibiting the production of a particular protein. Although not wishing to be limited by theory, Dougherty and Parks (Curr. Opin. Cell Biol. 7: 399 (1995)) have provided a model for the mechanism by which dsRNA can be used to reduce protein production. This model has recently been modified and expanded by Waterhouse et al. (Proc. Natl. Acad. Sci. 95: 13959 (1998)). This technology relies on the presence of dsRNA molecules-that contain a sequence that is essentially identical to the mRNA of the gene of interest, in this case an mRNA encoding a tropomyosin protein. Conveniently, the dsRNA can be produced in a single open reading frame in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules targeted against tropomyosin is well within the capacity of a person skilled in the art, particularly considering Dougherty and Parks (1995, supra), Waterhouse et al. (1998, supra), WO 99/32619, WO 99/53050, WO 99/49029, and WO 01/34815.

As used herein, the terms “small interfering RNA” (siRNA), and “RNAi” refer to homologous double stranded RNA (dsRNA) that specifically targets a gene product, thereby resulting in a null or hypomorphic phenotype. Specifically, the dsRNA comprises two short nucleotide sequences derived from the target RNA encoding PAC-1 and having self-complementarity such that they can anneal, and interfere with expression of a target gene, presumably at the post-transcriptional level. RNAi molecules are described by Fire et al., Nature 391, 806-811, 1998, and reviewed by Sharp, Genes & Development, 13, 139-141, 1999).

Preferred siRNA molecules comprise a nucleotide sequence that is identical to about 19-21 contiguous nucleotides of the target mRNA. Preferably, the target sequence is exon 1b of the TPM1 or TMP3 genes.

As exemplified herein, preferred siRNA against a tropomyosin encoding region comprises a 21-nucleotide sequence set forth in SEQ ID NO:16 or SEQ ID NO:17. For producing siRNA which include a stem loop structure from the exemplified siRNAs set forth in SEQ ID NOS:16 and 17, the sense and antisense strands are positioned such that they flank an intervening loop sequence. Preferred loop sequences will be known to those skilled in the art.

Small Molecule Inhibitors

Numerous organic molecules may be assayed for their ability to modulate the immune system. For example, within one embodiment of the invention suitable organic molecules may be selected either from a chemical library, wherein chemicals are assayed individually, or from combinatorial chemical libraries where multiple compounds are assayed at once, then deconvoluted to determine and isolate the most active compounds.

Representative examples of such combinatorial chemical libraries include those described by Agrafiotis et al., “System and method of automatically generating chemical compounds with desired properties,” U.S. Pat. No. 5,463,564; Armstrong, R. W., “Synthesis of combinatorial arrays of organic compounds through the use of multiple component combinatorial array syntheses,” WO 95/02566; Baldwin, J. J. et al., “Sulfonamide derivatives and their use,” WO 95/24186; Baldwin, J. J. et al., “Combinatorial dihydrobenzopyran library,” WO 95/30642; Brenner, S., “New kit for preparing combinatorial libraries.” WO 95/16918; Chenera, B. et al., “Preparation of library of resin-bound aromatic carbocyclic compounds,” WO 95/16712; Elliman, J. A., “Solid phase and combinatorial synthesis of benzodiazepine compounds on a solid support,” U.S. Pat. No. 5,288.514; Felder, E. et al., “Novel combinatorial compound libraries,” WO 95/16209: Lemer. R. et al., “Encoded combinatorial chemical libraries.” WO 93/20242; Pavia, M. R. et al., “A method for preparing and selecting pharmaceutically useful non-peptide compounds from a structurally diverse universal library,” WO 95/04277; Summerton, J. E. and D. D. Weller, “Morpholino-subunit combinatorial library and method,” U.S. Pat. No. 5,506,337; Holmes, C., “Methods for the Solid Phase Synthesis of Thiazolidinones, Metathiazanones, and Derivatives thereof,” WO 96/00148; Phillips, G. B. and G. P. Wei, “Solid-phase Synthesis of Benzimidazoles,” Tet. Letters 37:4887-90, 1996; Rubland, B. et al., “Solid-supported Combinatorial Synthesis of Structurally Diverse beta.-Lactams,” J. Amer. Chem. Soc. 111:253-4, 1996; Look, G. C. et al., “The Identification of Cyclooxygenase-1 Inhibitors from 4-Thiazolidinone Combinatorial Libraries,” Bioorg and Med. Chem. Letters 6:707-12, 1996.

Candidate compounds may be organic molecules, preferably small organic compounds having a molecular weight of more than 100 and less than about 2,500 daltons. Preferred small molecules are less than 2000, or less than 1500 or less than 1000 or less than 500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including saccharides, fatty acids, steroids, purines, pyrimidines, derivatives, structural analogs or combinations thereof.

In one embodiment, the present invention involves screening small molecule chemodiversity represented within libraries of parent and fractionated natural product extracts, to detect bioactive compounds as potential candidates for further characterization.

In one embodiment of the present invention, the candidate compound is obtained from expression products of a gene library, a low molecular weight compound library (such as the low molecular weight compound library of ChemBridge Research Laboratories), a cell extract, microorganism culture supernatant, bacterial cell components and the like. In one particular embodiment, the candidate compound is obtained from an extract of a strain of Enteropathogenic E. coli (EPEC).

Methods of Screening for Tropomyosin Agonists/Antagonists

Screening Protocol Based on Polarised Distribution of Tropomyosin

An example of a screening method in which the ability of a candidate compound to inhibit tropomyosin function may involve an analysis of the effect of the compound on polarised distribution of tropomyosin within a cell.

For example, cells expressing a labelled tropomyosin isoform of interest may be exposed to candidate compounds and monitored for the loss of polarised distribution of that tropomyosin isoform. The labelled tropomyosin isoform may be generated, for example, by expression of a fusion construct comprising tropomyosin linked to a fluorescent compound (such as the green fluorescent protein (GFP)) within the cell. Those skilled in the art would understand that other detectable labels may be used in this screening assay.

Alternatively, a sample of cells may be exposed to a candidate compound, and the distribution of the tropomyosin isoform of interest determined by antibody staining.

Screening Protocol Based on Expression of Tropomyosin

An example of a screening method in which the ability of a candidate compound to inhibit tropomyosin expression may involve the following steps:

(i) contacting a candidate compound with cells capable of expressing tropomyosin,

(ii) measuring the amount of expression of tropomyosin in the cells brought into contact with the candidate compound and comparing this amount of expression with the amount of expression (control amount of expression) of tropomyosin in the corresponding control cells not brought into contact with an investigational substance, and

(iii) selecting a candidate compound showing a reduced amount of expression of tropomyosin as compared with the amount of control expression on the basis of the result of the above step (ii).

The cells used in this screening method may be any cells that can express tropomyosin, irrespective of the difference between natural and recombinant genes. Moreover, the derivation of the tropomyosin is not particularly limited. The cells may be human derived, or may derive from mammals other than humans such as mice, or from other organisms. Examples of suitable human cells are hematopoietic cells including mast cells. Moreover, transformed cells that contain expression vectors comprising nucleic acid sequences that encode tropomyosin may also be used.

The conditions for allowing the candidate compound to come into contact with the cells that can express tropomyosin are not limited, but it is preferable to select from among culture conditions (temperature, pH, culture composition, etc.) which will not kill the applicable cells, and in which the tropomyosin genes can be expressed.

The term “reduced” refers not only the comparison with the control amount of expression, but also encompasses cases where no tropomyosin is expressed at all. Specifically, this includes circumstances wherein the amount of expression of tropomyosin is substantially zero.

The amount of expression of tropomyosin can be assessed either by measuring the amount of expression of a tropomyosin gene (mRNA) or by measuring the amount of a tropomyosin protein produced. In addition, the method to measure the amount of tropomyosin need not be a method to directly measure the amount of expression of gene (mRNA) or the amount of protein produced, but may be any method that reflects these.

Specifically, to measure the amount of expression of tropomyosin (detection and assay), the amount of expression of tropomyosin mRNA may be measured utilizing DNA array or well-known methods such as the Northern blot method, as well as the RT-PCR method that utilizes oligonucleotides having nucleotide sequences complementary to the nucleotide sequence of the applicable tropomyosin mRNA. Moreover, the amount of tropomyosin protein may be measured by implementing such well-known methods as the Western blot method utilizing an anti-tropomyosin antibody.

The measurement of the amount of expression of tropomyosin (detection and assay) may be implemented by measuring the activity of proteins derived from marker genes, using a cell line into which have been introduced fused genes comprising the marker genes such as reporter genes (e.g., luciferase genes, chloramphenicol-acetyltransferase genes, β-glucuronidase genes, β-galactosidase genes and aequorin genes) linked to the tropomyosin gene. Alternatively, the expression of tropomyosin can be measured in a genetically engineered cell wherein a reporter sequence is introduced into the tropomyosin gene by homologous recombination so that the tropomyosin product expressed from that gene is labelled with the reporter.

Screening Protocol Based on Binding of Tropomyosin to One or More of its Binding Partners

In one embodiment, tropomyosin agonists or antagonists are identified by screening for candidate compounds which interfere with the binding of tropomyosin to a tropomyosin binding partner. An examples of a suitable tropomyosin binding partner is actin.

Standard solid-phase ELISA assay formats are particularly useful for identifying antagonists of the protein-protein interaction. In accordance with this embodiment, one of the binding partners, e.g an actin filament, is immobilized on a solid matrix, such as, for example an array of polymeric pins or a glass support. Conveniently, the immobilized binding partner is a fusion polypeptide comprising Glutathione-S-transferase (GST; e.g. a CAP-actin fusion), wherein the GST moiety facilitates immobilization of the protein to the solid phase support. The second binding partner (e.g. tropomyosin) in solution is brought into physical relation with the immobilized protein to form a protein complex, which complex is detected using antibodies directed against the second binding partner. The antibodies are generally labelled with fluorescent molecules or conjugated to an enzyme (e.g. horseradish peroxidase), or alternatively, a second labelled antibody can be used that binds to the first antibody. Conveniently, the second binding partner is expressed as a fusion polypeptide with a FLAG or oligo-histidine peptide tag, or other suitable immunogenic peptide, wherein antibodies against the peptide tag are used to detect the binding partner. Alternatively, oligo-HIS tagged protein complexes can be detected by their binding to nickel-NTA resin (Qiagen), or FLAG-labeled protein complexes detected by their binding to FLAG M2 Affinity Gel (Kodak). It will be apparent to the skilled person that the assay format described herein is amenable to high throughput screening of samples, such as, for example, using a microarray of bound peptides or fusion proteins.

A two-hybrid assay as described in U.S. Pat. No. 6,316,223 may also be used to identify compounds that interfere with the binding of tropomyosin to one of its binding partners. The basic mechanism of this system is similar to the yeast two hybrid system. In the two-hybrid system, the binding partners are expressed as two distinct fusion proteins in a mammalian host cell. In adapting the standard two-hybrid screen to the present purpose, a first fusion protein consists of a DNA binding domain which is fused to one of the binding partners, and a second fusion protein consists of a transcriptional activation domain fused to the other binding partner. The DNA binding domain binds to an operator sequence which controls expression of one or more reporter genes. The transcriptional activation domain is recruited to the promoter through the functional interaction between binding partners. Subsequently, the transcriptional activation domain interacts with the basal transcription machinery of the cell, thereby activating expression of the reporter gene(s), the expression of which can be determined. Candidate bioactive agents that modulate the protein-protein interaction between the binding partners are identified by their ability to modulate transcription of the reporter gene(s) when incubated with the host cell. Antagonists will prevent or reduce reporter gene expression, while agonists will enhance reporter gene expression. In the case of small molecule modulators, these are added directly to the cell medium and reporter gene expression determined. On the other hand, peptide modulators are expressible from nucleic acid that is transfected into the host cell and reporter gene expression determined. In fact, whole peptide libraries can be screened in transfected cells.

Alternatively, reverse two hybrid screens, such as, for example, described by Vidal et al., Proc. Natl Acad. Sci USA 93, 10315-10320, 1996, may be employed to identify antagonist molecules. Reverse hybrid screens differ from froward screens supra in so far as they employ a counter-selectable reporter gene, such as for example, CYH2 or LYS2, to select against the protein-protein interaction. Cell survival or growth is reduced or prevented in the presence of a non-toxic substrate of the counter-selectable reporter gene product, which is converted by said gene product to a toxic compound. Accordingly, cells in which the protein-protein interaction of the invention does not occur, such as in the presence of an antagonist of said interaction, survive in the presence of the substrate, because it will not be converted to the toxic product. For example, a portion/fragment of tropomyosin that binds to actin is expressed as a DNA binding domain fusion, such as with the DNA binding domain of GAL4; and the portion of actin that binds tropomyosin is expressed as an appropriate transcription activation domain fusion polypeptide (e.g. with the GAL4 transcription activation domain). The fusion polypeptides are expressed in yeast in operable connection with the URA3 counter-selectable reporter gene, wherein expression of URA3 requires a physical relation between the GAL4 DNA binding domain and transcriptional activation domain. This physical relation is achieved, for example, by placing reporter gene expression under the control of a promoter comprising nucleotide sequences to which GAL4 binds. Cells in which the reporter gene is expressed do not grow in the presence of uracil and 5-fluororotic acid (5-FOA), because the 5-FOA is converted to a toxic compound. Candidate peptide inhibitor(s) are expressed in libraries of such cells, wherein cells that grow in the presence of uracil and 5-FOA are retained for further analysis, such as, for example, analysis of the nucleic acid encoding the candidate peptide inhibitor(s). Small molecules that antagonize the interaction are determined by incubating the cells in the presence of the small molecules and selecting cells that grow or survive of cells in the presence of uracil and 5-FOA.

Alternatively, a protein recruitment system, such as that described in U.S. Pat. No. 5,776,689 to Karin et al., may be used. In a standard protein recruitment system, a protein-protein interaction is detected in a cell by the recruitment of an effector protein, which is not a transcription factor, to a specific cell compartment. Upon translocation of the effector protein to the cell compartment, the effector protein activates a reporter molecule present in that compartment, wherein activation of the reporter molecule is detectable, for example, by cell viability, indicating the presence of a protein-protein interaction.

More specifically, the components of a protein recruitment system include a first expressible nucleic acid encoding a first fusion protein comprising the effector protein and one of the binding partners (e.g. actin or a portion thereof), and a second expressible nucleic acid molecule encoding a second fusion protein comprising a cell compartment localization domain and the other binding partner (e.g. tropomyosin or a portion thereof). A cell line or cell strain in which the activity of an endogenous effector protein is defective or absent (e.g. a yeast cell or other non-mammalian cell), is also required, so that, in the absence of the protein-protein interaction, the reporter molecule is not expressed.

A complex is formed between the fusion polypeptides as a consequence of the interaction between the binding partners, thereby directing translocation of the complex to the appropriate cell compartment mediated by the cell compartment localization domain (e.g. plasma membrane localization domain, nuclear localization domain, mitochondrial membrane localization domain, and the like), where the effector protein then activates the reporter molecule. Such a protein recruitment system can be practised in essentially any type of cell, including, for example, mammalian, avian, insect and bacterial cells, and using various effector protein/reporter molecule systems.

For example, a yeast cell based assay is performed, in which the interaction between tropomysoin and one or more of its binding partners results in the recruitment of a guanine nucleotide exchange factor (GEF or C3G) to the plasma membrane, wherein GEF or C3G activates a reporter molecule, such as Ras, thereby resulting in the survival of cells that otherwise would not survive under the particular cell culture conditions. Suitable cells for this purpose include, for example, Saccharomyces cerevisiae cdc25-2 cells, which grow at 36° C. only when a functional GEF is expressed therein, Petijean et al., Genetics 124, 797-806, 1990). Translocation of the GEF to the plasma membrane is facilitated by a plasma membrane localization domain. Activation of Ras is detected, for example, by measuring cyclic AMP levels in the cells using commercially available assay kits and/or reagents. To detect antagonists of the protein-protein interaction of the present invention, duplicate incubations are carried out in the presence of a test compound, or in the presence or absence of expression of a candidate antagonist peptide in the cell. Reduced survival or growth of cells in the presence of a candidate compound or candidate peptide indicates that the peptide or compound is an antagonist of the interaction between tropomysoin and one or more of its binding partners.

A “reverse” protein recruitment system is also contemplated, wherein modified survival or modified growth of the cells is contingent on the disruption of the protein-protein interaction by the candidate compound or candidate peptide. For example, NIH 3T3 cells that constitutively express activated Ras in the presence of GEF can be used, wherein the absence of cell transformation is indicative of disruption of the protein complex by a candidate compound or peptide. In contrast, NIH 3T3 cells that constitutively express activated Ras in the presence of GEF have a transformed phenotype (Aronheim et al., Cell. 78, 949-961, 1994)

In yet another embodiment, small molecules are tested for their ability to interfere with binding of tropomyosin to one of its binding partners, by an adaptation of plate agar diffusion assay described by Vidal and Endoh, TIBS 17, 374-381, 1999, which is incorporated herein by reference.

In a preferred embodiment of the invention the tropomyosin binding partner is selected from the group consisting of calponin (Childs et al. BBA 1121: 41-46, 1992), Cancinoembryonic antigen cell adhesion molecule 1 (CEACAM1) (Schumann et al., J. Biol. Chem. 276 (50):47421-33, 2001), endostatin (MacDonald et al. J. Biol. Chem. 276, 25190-25196, 2001), Enigma (Guy et al. FEBS letters 10: 1973-1984, 1999), Gelsolin (preferably sub-domain 2) Koepf and Burtnick FEBS 309(1): 56-58, 1992), S100A2 (Gimona et al. J. Cell Sci. 110: 611-621, 1997) and actin. In a further preferred embodiment, the tropomyosin binding partner is actin.

Screening Method Based on Myosin ATPase Activity

In an adaptation of the screening protocol based on binding of tropomyosin to one or more of its binding partners, the method involves the addition of myosin to the reaction mix and detection of myosin ATPase activity.

For example, a tropomyosin isoform may be incubated with actin filaments and specific myosins. Myosin ATPase activity is then measured in the presence of the candidate compounds. Under normal conditions, tropomyosin inhibits myosin ATPase activity. Accordingly, compounds that interact with tropmyosin and prevent this inhibitory activity will results in increased myosin ATPase activity. Such compounds may be selected for further screening and/or characterisation. Suitable positive control reactions may be performed without tropomyosin or with an inappropriate tropomyosin isoform to eliminate anti-myosin effects.

Methods for determining myosin ATPase activity that can be adapted for use in the present invention will be known to those skilled in the art. Examples of such assays are described in Zhao et al., Biochem. Biophys. Res Commun. 267(1):77-79, 2000; Westra et al., Archives of Physiology and Biochemistry 109:316-322, 2001; and Droft et al., Biochem J. 264:191-8, 1989.

Therapeutic Methods

The tropomyosin agonists or antagonists identified by the methods of the present invention can be used therapeutically for diseases caused by abnormal insertion, retention or function of cell surface proteins. The term “therapeutically” or as used herein in conjunction with the tropomyosin agonists or antagonists of the invention denotes both prophylactic as well as therapeutic administration. Thus, tropomyosin agonists/antagonists can be administered to high-risk patients in order to lessen the likelihood and/or severity of a disease or administered to patients already evidencing active disease.

Diseases or conditions of humans or other species which can be treated with agonists or antagonists of tropomyosin function include, but are not limited to: cystic fibrosis, multiple sclerosis, polycistic kidney disease, viral infection, bacterial infection, reperfusion injury, Menkes Disease, Wilson's Disease, diabetes, myotonic dystrophies, epilepsy or a mood disorder such as depression, bipolar disorder or dysthymic disorder.

Modes of Administration

In the case where the candidate compound is in the form of a low molecular weight compound, a peptide or a protein such as an antibody, the substance can be formulated into the ordinary pharmaceutical compositions (pharmaceutical preparations) which are generally used for such forms, and such compositions can be administered orally or parenterally. Generally speaking, the following dosage forms and methods of administration can be utilized

The dosage form includes such representative forms as solid preparations, e.g. tablets, pills, powders, fine powders, granules, and capsules, and liquid preparations, e.g. solutions, suspensions, emulsions, syrups, and elixirs. These forms can be classified by the route of administration into said oral dosage forms or various parenteral dosage forms such as transnasal preparations, transdermal preparations, rectal preparations (suppositories), sublingual preparations, vaginal preparations, injections (intravenous, intraarterial, intramuscular, subcutaneous, intradermal) and drip injections. The oral preparations., for instance, may for example be tablets, pills, powders, fine powders, granules, capsules, solutions, suspensions, emulsions, syrups, etc. and the rectal and vaginal preparations include tablets, pills, and capsules, among others. The transdermal preparations may not only be liquid preparations, such as lotions, but also be semi-solid preparations, such as creams, ointments, and so forth.

The injections may be made available in such forms as solutions, suspensions and emulsions; and as vehicles, sterilized water, water-propylene glycol, buffer solutions, and saline of 0.4 weight % concentration can be mentioned as examples. These injections, in such liquid forms, may be frozen or lyophilized. The latter products, obtained by lyophilization, are extemporaneously reconstituted with distilled water for injection or the like and administered. The above forms of pharmaceutical composition (pharmaceutical preparation) can be prepared by formulating the compound having tropomyosin inhibitory action and a pharmaceutically acceptable carrier in the manner established in the art. The pharmaceutically acceptable carrier includes various excipients, diluents, fillers, extenders, binders, disintegrators, wetting agents, lubricants, and dispersants, among others. Other additives which are commonly used in the art can also be formulated. Depending on the form of pharmaceutical composition to be produced, such additives can be judiciously selected from among various stabilizers, fungicides, buffers, thickeners, pH control agents, emulsifiers, suspending agents, antiseptics, flavors, colors, tonicity control or isotonizing agents, chelating agents and surfactants, among others.

The pharmaceutical composition in any of such forms can be administered by a route suited to the objective disease, target organ, and other factors. For example, it may be administered intravenously, intraarterially, subcutaneously, intradermally, intramuscularly or via airways. It may also be directly administered topically into the affected tissue or even orally or rectally.

The dosage and dosing schedule of such a pharmaceutical preparation vary with the dosage form, the disease or its symptoms, and the patient's age and body weight, among other factors, and cannot be stated in general terms. The usual dosage, in terms of the daily amount of the active ingredient for an adult human, may range from about 0.0001 mg to about 500 mg, preferably about 0.001 mg to about 100 mg, and this amount can be administered once a day or in a few divided doses daily.

When the substance having tropomyosin inhibitory activity is in the form of a polynucleotide such as an antisense compound, the composition may be provided in the form of a drug for gene therapy or a prophylactic drug. Recent years have witnessed a number of reports on the use of various genes, and gene therapy is by now an established technique.

The drug for gene therapy can be prepared by introducing the object polynucleotide into a vector or transfecting appropriate cells with the vector. The modality of administration to a patient is roughly divided into two modes, viz. The mode applicable to (1) the case in which a non-viral vector is used and the mode applicable to (2) the case in which a viral vector is used. Regarding the case in which a viral vector is used as said vector and the case in which a non-viral vector is used, respectively, both the method of preparing a drug for gene therapy and the method of administration are dealt with in detail in several books relating to experimental protocols [e.g. “Bessatsu Jikken Igaku, Idenshi Chiryo-no-Kosogijutsu (Supplement to Experimental Medicine, Fundamental Techniques of Gene Therapy), Yodosha, 1996; Bessatsu Jikken Igaku: Idenshi Donyu & Hatsugen Kaiseki Jikken-ho (Supplement to Experimental Medicine: Experimental Protocols for Gene Transfer & Expression Analysis), Yodosha, 1997; Japanese Society for Gene Therapy (ed.): Idenshi Chiryo Kaihatsu Kenkyn Handbook (Research Handbook for Development of Gene Therapies), NTS, 1999, etc.].

When using a non-viral vector, any expression vector capable of expressing the anti-tropomyosin nucleic acid may be used. Suitable examples include pCAGGS [Gene 108, 193-200(1991)], pBK-CMV, pcDNA 3.1, and pZeoSV (Invitrogen, Stratagene).

Transfer of a polynucleotide into the patient can be achieved by inserting the object polynucleotide into such a non-viral vector (expression vector) in the routine manner and administering the resulting recombinant expression vector. By so doing, the object polynucleotide can be introduced into the patient's cells or tissue.

More particularly, the method of introducing the polynucleotide into cells includes the calcium phosphate transfection (coprecipitation) technique and the DNA (polynucleotide) direct injection method using a glass microtube, among others.

The method of introducing a polynucleotide into a tissue includes the polynucleotide transfer technique using internal type liposomes or electrostatic type liposomes, the HVJ-liposome technique, the modified HVJ-liposome (HVJ-AVE liposome) technique, the receptor-mediated polynucleotide transfer technique, the technique which comprises transferring the polynucleotide along with a vehicle (metal particles) into cells with a particle gun, the naked-DNA direct transfer technique, and the transfer technique using a positively charged polymer, among others.

Suitable viral vectors include vectors derived from recombinant adenoviruses and retrovirus. Examples include vectors derived from DNA or RNA viruses such as detoxicated retrovirus, adenovirus, adeno-associated virus, herpesvirus, vaccinia virus, poxvirus, poliovirus, sindbis virus, Sendai virus, SV40, human immunodeficiency virus (HIV) and so forth. The adenovirus vector, in particular, is known to be by far higher in infection efficiency than other viral vectors and, from this point of view, the adenovirus vector is preferably used.

Transfer of the polynucleotide into the patient can be achieved by introducing the object polynucleotide into such a viral vector and infecting the desired cells with the recombinant virus obtained. In this manner, the object polynucleotide can be introduced into the cells.

The method of administering the thus-prepared drug for gene therapy to the patient includes the in vivo technique for introducing the drug for gene therapy directly into the body and the ex vivo technique which comprises withdrawing certain cells from a human body, introducing the drug for gene therapy into the cells in vitro and returning the cells into the human body [Nikkei Science, April, 1994 issue, 20-45; Pharmaceuticals Monthly, 36(1), 23-48, 1994; Supplement to Experimental Medicine, 12(15), 1994; Japanese Society for Gene Therapy (ed.): Research Handbook for Development of Gene Therapies, NTS, 19991]. For use in the prevention or treatment of an inflammatory disease to which the present invention is addressed, the drug is preferably introduced into the body by the in vivo technique.

When the in vivo method is used, the drug can be administered by a route suited to the object disease, target organ or the like. For example, it can be administered intravenously, intraarterially, subcutaneously or intramuscularly, for instance, or may be directly administered topically into the affected tissue.

The drug for gene therapy can be provided in a variety of pharmaceutical forms according to said routes of administration. In the case of an injectable form, for instance, an injection can be prepared by the per se established procedure, for example by dissolving the active ingredient polynucleotide in a solvent, such as a buffer solution, e.g. PBS, physiological saline, or sterile water, followed by sterilizing through a filter where necessary, and filling the solution into sterile vitals, Where necessary, this injection may be supplemented with the ordinary carrier or the like. In the case of liposomes such as HVJ-liposome, the drug can be provided in various liposome-entrapped preparations in such forms as suspensions, frozen preparations and centrifugally concentrated frozen preparations.

Furthermore, in order that the gene may be easily localized in the neighborhood of the affected site, a sustained-release preparation (eg. a minipellet) may be prepared and implanted near the affected site or the drug may be administered continuously and gradually to the affected site by means of an osmotic pump or the like.

The polynucleotide content of the drug for gene therapy can be judiciously adjusted according to the disease to be treated, the patient's age and body weight, and other factors but the usual dosage in terms of each polynucleotide is about 0.0001.about. about 100 mg. preferably about 0.001.about. about 10 mg. This amount is preferably administered several days or a few months apart.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the putpose of providing a context for the present invention. It is not to be taken as an admission that any or all of these matters form part of the prior art base or were common general knowledge in the field relevant to the present invention as it existed in Australia before the priority date of each claim of this application.

The present invention will now be illustrated by the following Examples, which are not intended to be limiting in any way.

Experimental Details

Materials and Methods

Reagents and Antibodies

Cytochalasin D, forskolin, 6-Methoxyquinolinium 1-acetic acid ethyl ester (MQAE), nocodazole, 3′3′5′5′ Tetra methyl benzidine, 1,4-diazabicyclo[2.2.2.]octane (DABCO), poly-D-lysine and 1% collagen were purchased from Sigma (St. Louis, Mo., U.S.A.). Lipofectin reagent and antisense oligonucleotides were purchased from Invitrogen (Mulgrave, Vic, Australia). Jasplakinolide was purchased from Bio Scientific (Gymea, N.S.W., Australia). Nitroblue tetrazolium chloride and 5-bromo-4-chloro-3-indolylphosphate p-toluidine salt (NBT and BCIP), tissue culture medium and reagents were purchased from Life Technologies (Mulgrave, Vic, Australia). The bicinchoninic acid (BCA) protein assay kit was purchased from Pierce (Rockford Ill., U.S.A.). Thermanox coverslips and glass chamber slides were purchased from Medos (Mt Waverley, Vic, Australia). Tissue culture plasticware was purchased from Interpath (Morrisville N.C. U.S.A.). Western Lightening™ chemiluminescence reagent was purchased from Perkin Elmer Life Sciences Inc (Boston, Mass., U.S.A.).

Rhodamine Red X conjugate and rhodamine goat anti-sheep IgG were from Jackson Immunoresearch (West Grove, Pa., U.S.A). Horse radish peroxidase (HRP) anti-mouse and anti-rabbit IgG were from Amersham Life Sciences (Buckinghamshire, U.K.). The mouse monoclonal Tm antibodies 311 and the secondary antibody fluorescein isothiocyanate (FITC)-Donkey anti-mouse were from Sigma Aldrich (St. Louis, Mo., U.S.A.). Tm antibody CG3 was a gift from J. C. Lin (Univ. of Iowa, Iowa, U.S.A.). CFTR antibody (MA1-935) was from Affinity Bioreagents Inc. (Golden, Colo., USA). The mouse monoclonal anti-human, c-terminus specific, CFTR antibody was from Bio Scientific (Gymea, N.S.W., Australia).

Cell Culture

Human T84 colonic carcinoma cells were seeded onto 2 chamber glass slides, 24 or 96 well plates or glass coverslips coated with poly-D-lysine and 1% collagen. The T84 cells were obtained from the American Tissue Culture Laboratory (passage 60) and as a kind gift from Kim Barrett (San Diego, U.S.A.) (passage 20). During the course of the research, they were subcultured to passage 80 and 30 respectively. T84 cells were cultured using the method of Li et al (Li et al., 1999, Infection & Immunity 67, 5938-5945).

The viability of T84 cells following treatments was assessed using a trypan blue exclusion assay. Post-treatment, the T84 cell monolayers were washed gently with PBS and stained with 1% trypan blue for 10 minutes. The cells were examined immediately by phase contrast microscopy. Treated and control monolayers were compared by counting the number of cells with trypan blue uptake in random microscopy fields.

Immunofluorescence Analysis

Cells were washed in 2% foetal bovine serum (FBS) in phosphate buffered saline (PBS) then fixed with 4% paraformaldehyde. They were permeabilised with 100% methanol, chilled to −80° C., for 20 minutes. Cells were incubated at room temperature with primary and secondary antibodies for 1 hour with washes performed with 2% FBS in PBS 3 times for 10 minutes after each incubation. Coverslips were mounted onto the slides with the anti-fade reagent DABCO.

Fluorescence Microscopy

Fluorescence was examined with a confocal laser scanning microscope (Leica Microsystems, Wetzler, Germany) using a 63× oil emersion objective. The distribution of fluorophores was measured by scanning at 488 nm for FITC and 568 nm for rhodamine using 8 line averages to eliminate noise. Images were taken in the vertical (xz) and horizontal (xy) plane. Images in the horizontal plane were constructed by overlaying sections taken at 1 μm steps from the apical to the basal region of the cells.

The pixel intensity of Tm antibody staining was measured on images obtained by confocal microscopy. Measurements were made across the apical region and across the central region of monolayers and averaged to obtain the mean pixel intensity for individual monolayers. The distribution of antibody staining within individual monolayers is described as the ratio of the mean pixel intensity in the apical region to the mean pixel intensity in the central region of that monolayer. To determine the relative distribution of αf9d and 311 antibody staining, the apical:central mean pixel intensity ratios for αf9d and 311 were compared in co-stained monolayers, using Student t-test for paired samples.

Antibody Staining of Histological Specimens

Rat duodenal tissue specimens were fixed in 4% paraformaldehyde saline and stored in 70% ethanol at 4° C. until embedded in paraffin. Sections were dewaxed in xylol and rehydrated stepwise in graded ethanols (100%, 100%, 70%, water). Antigen retrieval was performed by boiling the specimens in 1× citrate buffer (10× citrate buffer: 5 g/l EDTA, 2.5g /l Tris base and 3.2 g/l tri-sodium citrate; pH 8.0), microwaving on high for 12 minutes then allowing to cool. The specimens were washed twice in PBS and blocked with 10% serum in PBS for 10 minutes. Primary antibodies were then applied overnight at room temperature. Specimens were washed twice in PBS for 5 minutes prior to application of the secondary antibodies. Secondary antibodies were applied for 1 hour after which the specimens were washed once in PBS for 5 minutes and once with alkaline phosphate buffer (10 mls of 0.1M tris pH 9.5, 5 mls 1M MgCl and 2 mls of 5M NaCl) for 5 minutes. The substrate containing NBT and BCIP was then applied for 40 to 60 minutes after which specimens were washed once with PBS for 5 minutes. Specimens were then counterstained with Nuclear Fast Red for 1 minute after which they were rinsed twice in distilled water, dehydrated in increasing grades of ethanol (70%, 100%, 100%, 100%), cleared with xylol and coversliped.

Cell Treatment with Jasplakinolide, Cytochalasin and Nocodazole During Monlayer Formation

Epithelial cell monolayers were trypsinalised using trypsin/EDTA and centrifuged to form a cell pellet. The cells were then resuspended in medium containing either 1 μM jasplakinolide, 20 μM cytochalasin D or 33 μM nocodazole and seeded into poly-d-lysine and collagen coated glass chamber slides. The developing monolayers were then fixed and stained at 10 minutes after seeding. The effect of nocodazole on microtubules was confirmed by staining with antibodies to β tubulin and comparing with untreated cells. Mature T84 cell monolayers were treated with medium containing 20μM cytochalasin D for 3 hours then fixed and stained. Immunofluorescence analysis was then performed as described above.

Antisense Oligonucleotides

The sequence of the antisense and nonsense phosphorothioated oligonucleotides to Tm5a and Tm5b were 5′-CAC CGC CUC CAG CGA GCT (SEQ ID NO:14) and 5′-GCT CCA GCC ACG CCG ACT (SEQ ID NO:15) respectively. These were designed from the exon 1b sequence of the human αTMfast gene (Novy et al., 1993, Cell Motility & the Cytoskeleton 25, 267-281). T84 cell monolayers were grown to confluence on coverslips, in glass chamber slides or 24 well plates. The oligonucleotides were applied at a concentration of 2 μM with Lipofectin Reagent at 10 μg/ml according to the manufacturers instructions. The T84 cell monolayers were then incubated with the oligonucleotide for 24 hours at 37° C. in 5% CO₂ after which time they were used for experiments that required oligonucleotide pretreatment.

Immunoblot Analysis of Tropomyosin Isoforms

Proteins were extracted from T84 cells using the method of Wessel and Flugge (Wessel and Flugge, 1984). Western blot was performed as described (Percival et al., 2000, Cell Motility & the Cytoskeleton 47, 189-208). In brief, proteins were fractionated by SDS-PAGE using 15% low bis acrylamide gels, transferred to polyvinylidene difluoride membranes and probed with Tm antibodies. Bound antibody was detected using HRP-conjugated goat anti-rabbit or goat anti-mouse IgG. The bands were detected using Western Lightening™ chemiluminescence reagent and exposure to x-ray film.

Protein expression was measured as the density of protein bands on Western blot autoradiographs using the computer program Molecular Analyst (Version 1.5, Bio Rad Laboratories, Calif., U.S.A.). The protein band density is reported as the protein band density normalised to the protein band density for the control group within individual experiments. To determine the effects of treatment, normalised protein band density was compared to the null hypothesis value of 1 by the one-sided Student t test.

MQAE Chloride Efflux Assay

T84 cell monolayers, cultured on 24 or 96 well plates were incubated in medium containing 10 mM MQAE for 16 hours. The monolayers were then washed 3 times in chloride buffer (2.4 mM Na₂HPO₄, 0.6 mM NaH₂PO₄, 1 mM K₂SO₄, 1 mM MgSO₄, 3.4 mM KCl, 124.6 mM NaCl, 1 mM CaCl₂, 10 mM glucose and 10 mM HEPES ). T84 cell monolayers were stimulated with forskolin by incubating with chloride buffer containing 10 μM forskolin for 10 minutes, after which the chloride buffer was removed and replaced with chloride free buffer (2.4 mM Na₂HPO₄, 0.6 mM NaH₂PO₄, 1 mM K₂SO₄, 1 mM MgSO₄, 3.4 mM KNO₃, 1 mM Ca(NO₃)₂, 124.6 mM NaNO₃, 10 mM glucose and 10 mM HEPES) containing 10 μM forskolin. Repetitive fluorescence measurements were initiated immediately using a fluorescence plate reader (excitation, λ-360 nm; emission, λ-460 nm). Measurements were performed every 30 to 60 seconds for 15 minutes.

Chloride efflux was measured as the percentage increase in fluorescence between baseline and the specified time point. The percentage increase in fluorescence was normalised within experiments to the mean percentage increase in fluorescence in the control group in that experiment. To determine the effects of treatments, the normalised percentage increase in fluorescence was compared between groups. Two group comparisons were made using the Student t test.

Enzyme Linked Surface CFTR Assay

T84 cell monolayers cultured on collagen coated glass coverslips were incubated in either chloride buffer with 10 μM forskolin or chloride buffer only for 30 minutes at 37° C. in 5% CO2 then fixed with 4% paraformaldehyde for 20 minutes at 4° C. The T84 cell monolayers were incubated for 1 hour firstly with CFTR (MA1-935) antibody (Walker et al., 1995) diluted 1:500 followed by HRP anti-mouse IgG diluted 1:1000. T84 cell monolayers were blocked prior to each incubation for 10 minutes in PBS containing 10% FBS and washed following each incubation 4 times in PBS. The coverslips were then placed into a clean 24 well plate and incubated for 30 minutes with 500 μl of 3′3′5′5′ Tetra methyl benzidine. The supernatant from each well was transferred to a cuvette and absorbance determined at 655 nm in a Beckman DU650 spectrophotometer. Absorbance was also determined at 655 nm for primary antibody negative controls and that amount was subtracted from the absorbance in primary antibody positive monolayers to determine their assay result.

The CFTR surface expression is reported as the absorbance measured at 655 nm, normalised to the mean absorbance for the control group within individual experiments. To determine the effects of treatment, normalised absorbance at 655 nm was compared between groups. Two group comparisons were made using the Student t test.

### EXAMPLE 1

### Tropomyosin Gene Expression and Antibody Specfficity in T84 Cells

Tm proteins are encoded by 4 distinct genes. The antibodies used in this study were capable of detecting specific isoforms that are produced from 3 Tm genes. The exon/intron structure of these genes is shown in FIG. 1. The αf9d antibody detects Tm 1, 2, 3, 5a, 5b and 6 (Schevzov et al., 1997, Molecular & Cellular Neurosciences 8, 439-454). The 311 antibody detects a subset of Tms detected by the αf9d antibody, namely Tm 1, 2, 3, and 6. The CG3 antibody detects Tm5NM1-11 (Novy et al., 1993, Cell Motility & the Cytoskeleton 25, 267-281; Dufour et al., 1998, Journal of Biological Chemistry 273, 18547-18555).

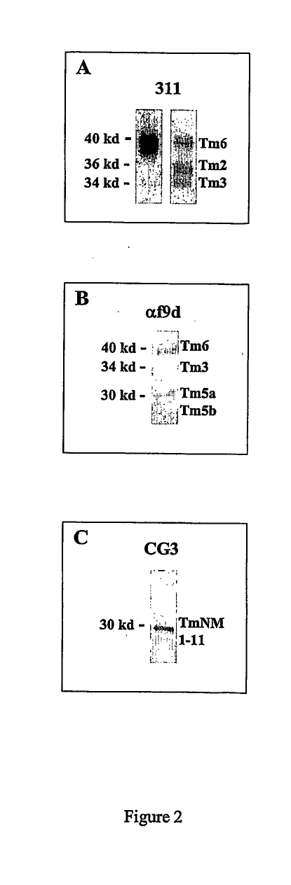

In human fibroblasts, the 311 antibody detected 3 bands. Bands were seen at 40, 36 and 34 kDa corresponding to Tm 6, 2 and 3 respectively (FIG. 2A). In T84 cells, the 311 antibody detected only the bands at 40 and 34 kDa corresponding to Tm 6 and 3 (FIG. 2A). The αf9d antibody detected 4 bands in T84 cells with bands seen at 40 and 34 kDa corresponding to Tm6 and Tm3 and a double band at 30 kDa corresponding to Tm's 5a and 5b (FIG. 2B). The CG3 antibody detected a single band at 30 kDa corresponding to co-migrating Tm5NM isoforms (FIG. 2C).

### EXAMPLE 2

### T84 Cell Monolayers Express a Polarised Distribution of Tm5a and Tm5b

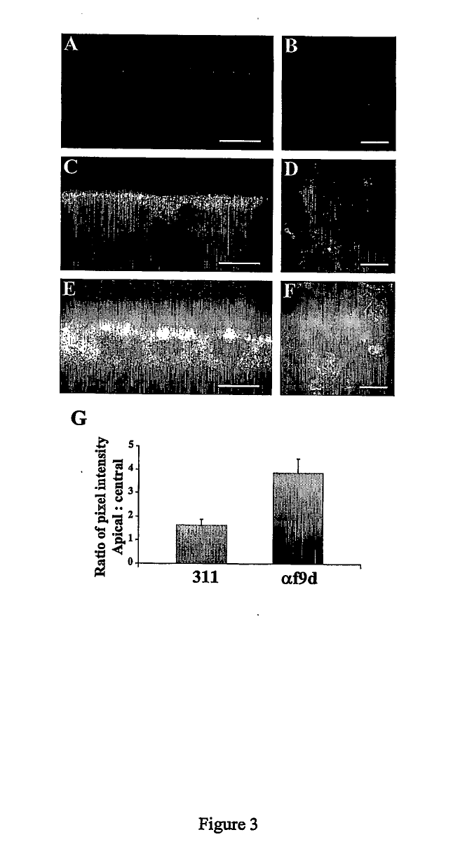

To determine the distribution of the separate microfilament populations in T84 cells, eight monolayers stained with each antibody were examined in both the vertical and horizontal planes by confocal microscopy. Representative images are presented in FIG. 3. The αf9d antibody, which detects Tm 3, 5a, 5b and 6 was found to have predominant staining at the apical pole of the cells (FIG. 3A). However, the 311 antibody, which recognises Tm 3 and 6, was found to have a more uniform distribution from the apical to basal pole of the same cells (FIG. 3C). This differential staining pattern can only be explained by the existence of a highly polarised distribution of the two isoforms detected by αf9d which are not detected by 311 (i.e. Tm5a and Tm5b). We therefore conclude that Tm5a and Tm5b are highly enriched at the apical surface. The antibody CG3, which stains Tm5NM 1-11, was distributed throughout the cell (FIG. 3E).

In sections through the epithelial monolayer taken in the horizontal plane, the distribution of αf9d (FIG. 3B) and 311 (FIG. 3D) were found to be associated with the lateral cell membrane and a paucity of staining was seen in the cytoplasm. CG3 was found to be located in the cytoplasm surrounding the cell nucleus (FIG. 3F).

The quantitative analysis of the relative distribution of αf9d and 311 antibody staining, which is depicted in FIG. 3G, confirmed the qualitative differences described above. The mean ratio of apical to central pixel intensity for the αf9d antibody was significantly higher than that of the 311 antibody (3.88±0.60 vs 1.64±0.23; p<0.001).

### EXAMPLE 3

### The Polarised Distribution of Specific Microfilament Populations Varies with Epithelial Cell Differentiation in the Rat Duodenum

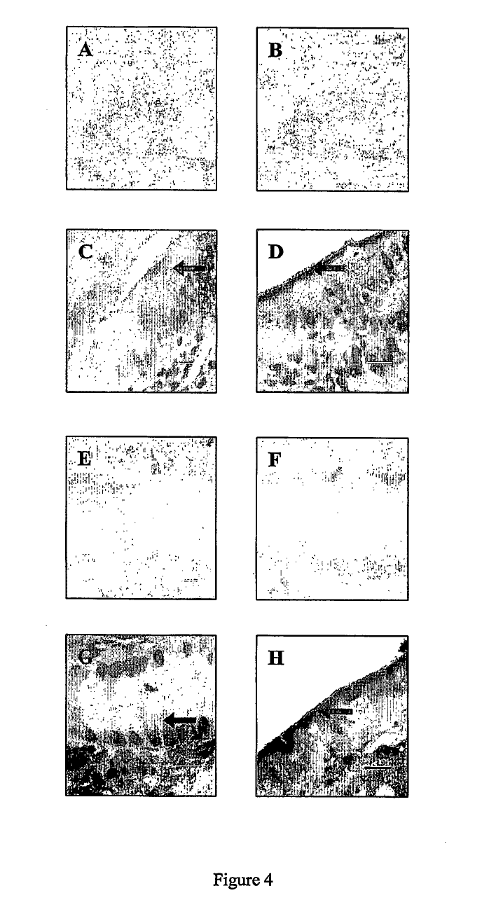

To determine whether the distribution of Tm isoforms observed in T84 cells differed from that seen in vivo in both crypt and villus gastrointestinal epithelial cells, 6 rat duodenal tissue specimens were stained for Tm isoforms and examined with brightfield microscopy. Representative sections are shown in FIG. 4. Staining with the αf9d antibody showed diffuse staining in the crypt epithelium (FIG. 4C arrow). The staining in the more differentiated villus epithelium was highly enriched in the apical region but also seen throughout the cytoplasm (FIG. 4D arrow). Staining with the 311 antibody (Tm 3 and 6) was sparse in the crypt epithelium (FIG. 4E). In the villus epithelium, the blue staining was seen in a circular area located above the nucleus (FIG. 4F). Staining with the CG3 antibody (Tm5NM 1-11) showed a similar distribution to that seen with the αf9d antibody. In the crypt epithelial cells, the staining was diffuse throughout the cell (FIG. 4G), whereas in the villus epithelial cells there was strong enrichment of staining in the apical region (FIG. 4H). In the goblet cells, which are predominantly found in the crypts, the staining was diffuse outside of the characteristic mucinous vacuole (FIG. 4G).

These results demonstrate that Tm isoforms are polarised in the more differentiated villus epithelial cells and are not polarised in the less differentiated crypt epithelial cells. Importantly, the relative distributions of αf9d antibody and 311 antibody staining in duodenal villus epithelial cells indicates that Tm5a and Tm5b are polarised in vivo in the same way they are polarised in the T84 cell model.

### EXAMPLE 4

### Polarised Distribution of Specific Microfilament Populations Occurs in the Early Phases of Monolayer Formation

The time sequence over which the polarised distribution of αf9d staining occurs was examined at 10 minutes, 1, 2 and 24 hours after seeding T84 cells. Three experiments were performed for each time point. Tm isoform expression was examined by performing Western blots on protein extracted from T84 cells collected at 1, 2, 4 and 24 hours and 7 days post seeding. Three experiments were performed at each time point.

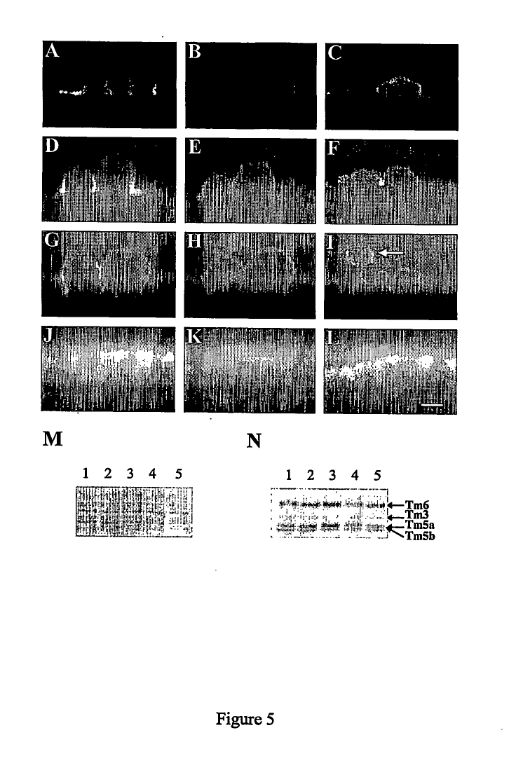

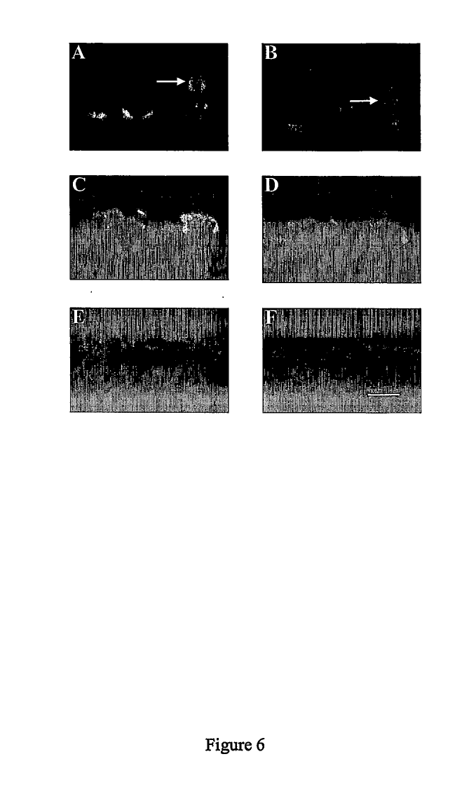

Representative confocal microscopy images are shown in FIG. 5. In T84 cells seen in suspension, αf9d, 311 and CG3 (FIG. 6A, 6B and 5I, arrow and data not shown) antibody staining was circumferential. Ten minutes after seeding (5A-C), the T84 cells were generally observed to make cell-cell contact and cell-slide contact. For all antibodies, there was reduced staining at the site of cell-slide contact at this initial time point although staining is more apparent for CG3 and 311 than for αf9d. Further, αf9d antibody staining appeared to be limited to the free surface while 311 antibody staining was more prominent at the site of cell-cell contact. In contrast, CG3 antibody staining was more evenly distributed over both the free surface and sites of cell-cell contact. Over time, the distribution of αf9d staining was basically unchanged (5E, H and K) with enriched staining at the free surface (reflecting Tm5a and Tm5b) and lower level diffuse staining resembling that of 311 (reflecting Tm6 and Tm3). In contrast, the distribution of 311 antibody staining (5D, G and J) and CG3 antibody staining (5F, I and L) both became more evenly distributed throughout the cell to include all surfaces and the cytoplasm.

Western blot analysis revealed that T84 cells collected 2 and 4 hours post seeding had a slightly increased expression of Tm 6 and 5a compared with cells collected at 24 hours and 7 days post seeding (FIG. 5N). The changes in levels of these isoforms cannot account for the alterations in staining of the αf9d and 311 antibodies. These alterations in isoform distribution are therefore most likely to result from altered targeting of these proteins.

### EXAMPLE 5

### The Early Polarised Distribution of Tm5a and Tm5b does not Involve Filament Turnover and is not Microtubule Dependent

Possible mechanisms for the development of microfilament polarisation were explored by drug manipulation of the cytoskeleton during seeding. Jasplakinolide was used to stabilise actin filaments, cytochalasin D was used to fragment actin filaments and nocodazole was used to disrupt microtubules. These drugs were applied to the T84 cells while they were in suspension, 10 minutes prior to plating. Cells were examined 10 minutes after plating.

Pretreatment of T84 cells with jasplakinolide prior to seeding altered cell morphology. The T84 cells had a flattened appearance (FIG. 6A) compared with untreated cells (FIG. 5A). In the T84 cells pretreated with jasplaldnolide, the distribution of both αf9d (FIG. 6B) and 311 (FIG. 6A) antibody staining 10 minutes post-seeding was similar to that of the control T84 cells (5A and 5B). The distribution of αf9d antibody remained apical, whilst the 311 antibody distribution appeared more prominent at the sites of cell-cell contact. Pretreatment of T84 cells with cytochalasin D prior to seeding prevented cell-slide adherence and no images were obtained. However, treatnent of established monolayers with cytochalasin D eliminated the polarised distribution of αf9d staining indicating that its maintenance requires an intact actin cytoskeleton (FIG. 6F)

Pretreatment of the T84 cells with nocodazole prior to seeding altered cell morphology. The T84 cells changed from having a curved surface (FIG. 5A) to having an irregular appearance (FIG. 6C). The distribution of the αf9d (FIG. 6D) antibody staining in nocodazole treated T84 cells 10 minutes post-seeding was similar to that of untreated T84 cells. Staining with the 311 antibody appeared similar to that of the αf9d antibody with enrichment at the apical surface and paucity of staining at the site of cell contact with the slide (FIG. 6C). Staining for β tubulin confirmed that nocodazole had disrupted normal microtubular structure (Data not shown).

These results suggest that the early polarisation of Tm5a and Tm5b does not involve filament turnover because the actin stabilising agent jasplakinolide did not affect the early development of polarisation. In addition, intact microtubules are not required as polarisation of Tm5a and Tm5b occurred despite microtubular disruption with nocodazole. However, microtubules may be involved in relocation of Tm3 and Tm6 to sites of cell-cell contact or their stabilisation at that site.

### EXAMPLE 6

### Tm5a and Tm5b Co-Localise with Membrane Inserted CFTR but not CFTR Contained in Sub-Apical Vesicles

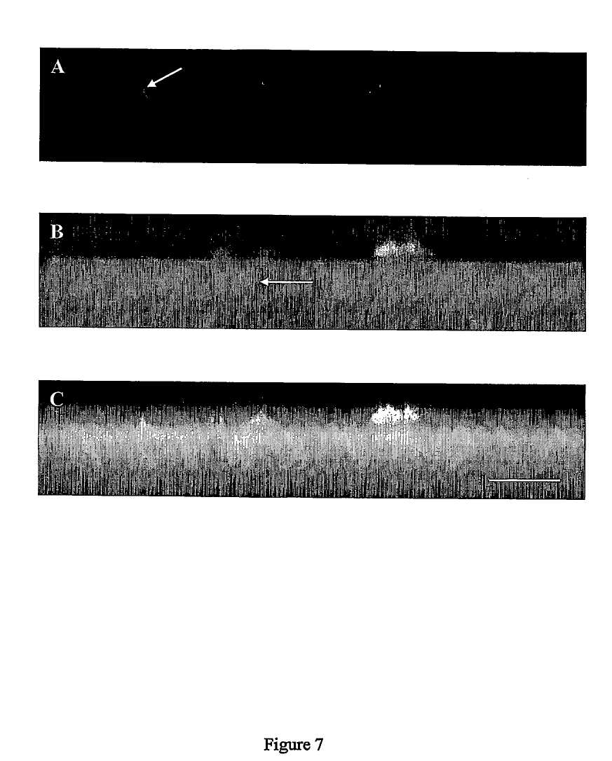

Staining of T84 cells with the CFTR antibody (FIG. 7B) demonstrated variable expression of CFTR. CFTR was seen in two forms. Some cells demonstrated prominent apical staining, with the CFTR appearing to protrude at the apical membrane. CFTR was also seen as smaller dots (FIG. 7B, arrow) located in the cell cytoplasm, giving the appearance of a location within a vesicle-like structure. Co-staining with the αf9d antibody revealed the typical polarised appearance at apical enrichment in addition to sites of very intense apical staining projecting above the surrounding apical surface. These highly enriched sites of αf9d staining were coincident with the sites of membrane incorporation of CFTR (FIG. 7C). The Tms therefore appeared to be incorporated into a structure associated with CFTR. All sites of membrane staining of CFTR were associated with these intense sites of αf9d staining. However, not all intense sites of αf9d staining showed significant CFTR staining suggesting that αf9d antibody staining is associated with sites available for CFTR insertion. The αf9d antibody did not co-localise with the CFTR contained within the cytoplasmic vesicle-like structures.

### EXAMPLE 7

### Tm5a and Tm5b Antisense Oligonucleotides Alter the Intensity of Apical Staining of αf9d in T84 Cell Monolayers

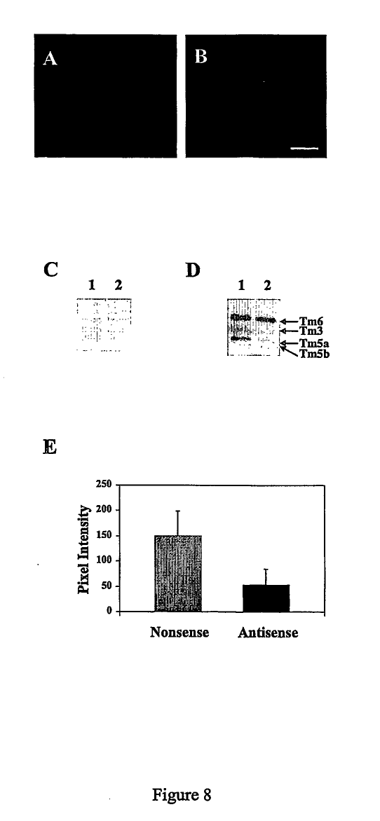

Previous studies have shown that cytochalasin D induced disruption of actin filaments increase chloride currents through CFTR (Prat et al., 1995, American Journal of Physiology 268, C1552-C1561) and we have observed that cytochalasin D also disrupts the polarised distribution of αf9d staining (FIG. 6F). We therefore reasoned that the actin filaments marked by αf9d, which colocalise with CFTR might inhibit chloride secretion by CFTR. To test this, we treated T84 cell monolayers with an antisense oligonucleotide or a scrambled nonsense control. Western blot analysis showed a substantial reduction in Tm5a and Tm5b levels after 24 hours exposure to the oligonucleotide (FIG. 8D). Relative to nonsense treatment, the antisense produced a mean reduction in Tm5a and Tm5b levels of 54±13% (p=0.02).

The treatment of T84 cultures with antisense oligonucleotide eliminated the polarised distribution of αf9d staining, which became largely even throughout the cell (FIG. 8B). In contrast, the nonsense oligonucleotide had essentially no effect on the distribution of staining with αf9d (FIG. 8A). The redistribution of staining induced by the antisense oligonucleotide was paralleled by a reduction in pixel intensity of αf9d staining at the apical surface. T84 cell monolayers treated in parallel with these oligonucleotides resulted in less apical pixel intensity in antisense cultures compared with nonsense cultures (FIG. 8E). This is consistent with a decrease in the level of polarised Tms detected by the αf9d antibody.

In conclusion, treatment with an antisense oligonucleotide to exon 1b of the a fast gene resulted in a significant reduction in apical staining with the αf9d antibody. These results also confirm that the prominent apical αf9d antibody staining in untreated T84 cell monolayers is due to a polarised distribution of Tm5a and Tm5b.

### EXAMPLE 8

### Tm5a and Tm5b Antisense Oligonucleotides Increase CFTR Surface Expression and Chloride Efflux

Antisense reductions of Tm5a and Tm5b levels and elimination of the polarised distribution of αf9d staining provided the opportunity to assess the role of these molecules in CFTR surface expression. This revealed a 50% increase in antisense compared with nonsense controls (1.49±0.78 vs 1±0.42; p<0.001). This suggests that the presence of Tm5a and Tm5b is acting either as a barrier to CFTR insertion into the apical membrane or retention of CFTR in the membrane.

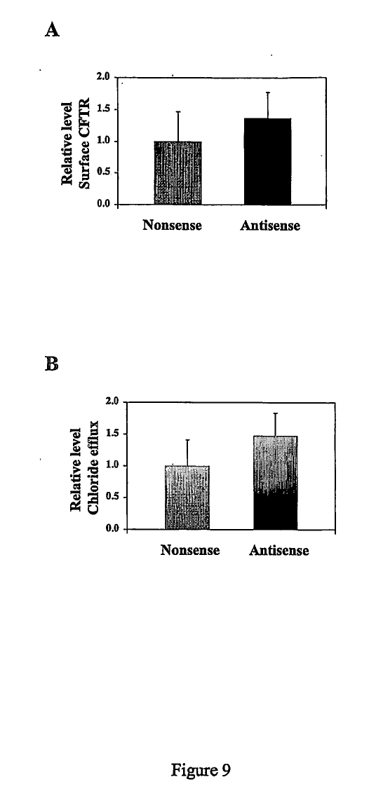

The increase in CFTR surface expression was paralleled by an increase in chloride efflux from antisense treated cells. In total, 21 T84 cell monolayers were treated with 2 μM antisense for 24 hours and were compared with 21 T84 cell monolayers treated with 2 μM nonsense for 24 hours. Following antisense and nonsense treatment, an MQAE chloride efflux assay was performed. The results are depicted in FIG. 9B. The T84 cell monolayers treated with antisense had significantly higher relative fluorescence measurement than T84 cell monolayers treated with nonsense after 15 minutes of 10 μM forskolin stimulation (1.47±0.41 vs 1±0.36; p<0.001).

### EXAMPLE 9

### Microtubule Disruption has no Effect on CFTR Surface Expression in T84 Cell Monolayers

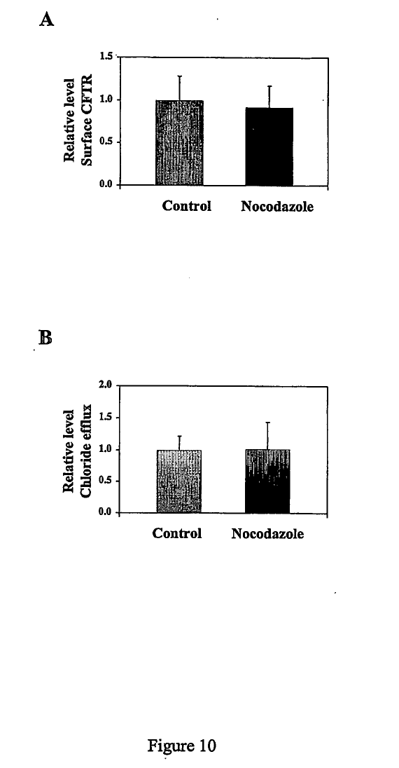

The impact on CFTR surface levels and chloride efflux by antisense treatment of Tms was not paralleled by disruption of microtubules. Incubation of T84 cells with nocodazole failed to elicit any significant change in either CFTR surface expression (FIG. 10A) nor chloride efflux (FIG. 10B). We conclude that these parameters are sensitive to disruption of the microfilament but not microtubule systems when assayed under short-term conditions. This correlates well with a more important role for actin filaments rather than microtubules in regulating the insertion of vesicle cargo's into the apical membrane or their retention.

### EXAMPLE 10

### The effect of Enteropathogenic E. coli (EPEC) Infection on the Actin Cytoskeleton

Enteropathogenic E. coli (EPEC) is responsible for up to 17% of gastroenteritis in children from Australian aboriginal communities. The mechanism by which it causes diarrhoea is unclear but increased chloride secretion has been implicated in animal models. We have previously demonstrated, in a cell culture model, that EPEC infection causes a reduction in epithelial cell chloride secretion through CFTR chloride channels and induces a redistribution of tropomyosin 5a and 5b isoforms within the epithelial cell's cytoskeleton. The function of these tropomyosins is unknown, but we have demonstrated that they are co-localised with CFTR chloride channels in the apical membrane.

The aim of this experiment was to examine the mechanism by which EPEC infection alters chloride secretion through CFTR chloride channels.

Cultured T84 colon cancer cell monolayers grown in collagen coated 24 well plates or on plastic coverslips were used as a model of the gastrointestinal epithelium. Incubation of monolayers inoculated with EPEC (104 organisms/well) for 6-9 hours was used to model EPEC infection and was compared with HB101, a non-pathogenic control. Antisense oligonucleotides were used to reduce tropomyosin 5a and 5b expression. An immuno-colourimetric assay was used to assess CFTR surface expression and intra-cellular MQAE fluorescence was used to assess chloride efflux.

The results showed that CFTR expression was increased (Mean increase: 153%; 95% CI: 100%, 205%; p<0.601) but chloride secretion was decreased by EPEC infection compared with HB101 (Mean decrease: 37%; 95% CI: 8%, 66%; p=0.014).

The redistribution of tropomyosin 5a and 5b may be causally related to the increased CFTR insertion in the apical membrane found with EPEC infection. Tropomyosin 5a and 5b containing filaments may provide a barrier to insertion of CFTR or retention in the apical membrane of gastrointestinal epithelial cells. The decrease in chloride secretion in the presence of elevated membrane CFTR suggests that EPEC can also inhibit CFTR chloride channel function. Diarrhoea may occur in EPEC infection as a rebound phenomenon following recovery of CFTR function in the presence of increased surface expression.

These results suggest that EPEC contains a compound that is capable of inhibiting the location or function of TM5a and TM5b. EPEC may therefore be a useful source of material for use in the screening assays described herein.

Discussion

Tropomyosin Isoform Sorting in Establishing Epithelial Cellpolarity

The development of polarisation with the creation of specialised functional domains is necessary for normal epithelial cell function. Central to the process of epithelial cell polarisation is the sorting, transport and insertion into the membrane of proteins, which give the domains their function (Yeaman et al., 1999, Physiological Reviews 79, 73-98). The role of the cell cytoskeleton, in particular the actin microfilament system, in this process is not clear. The rapid generation of actin cytoskeletal domains raises the possibility that cytoskeletal polarisation may be required for the development of functional polarity, particularly sorting and movement of proteins to specific membrane domains.

The findings in this study further support a role for microfilaments in the development of epithelial cell polarity and the polarised delivery of membrane proteins. We found that specific Tm isoforms polarise rapidly during monolayer development. The same isoforms were also found to regulate the insertion of CFTR and/or its retention in the apical membrane.

Mechanisms of Isoform Sorting

Drugs that interact with the cytoskeleton are widely used to examine cellular processes. By using these techniques, we were able to examine the mechanism by which epithelial cells were rapidly able to sort microfilaments. In the developing monolayer Jasplakinolide, a drug that prevents the breakdown and turnover of actin filaments, did not affect the early polarisation of Tm5a and Tm5b. In mature monolayers cytochalasin D, which breaks up actin filaments, disrupted the polarised distribution of Tm5a and Tm5b. Thus we can conclude that intact microfilaments are required for both the development of polarisation of Tm isoforms as well as the maintenance of this polarity. In addition the Tm isoforms form part of a higher order structure involving actin filaments rather than existing as isolated molecules.

The sorting of Tm isoforms found in our study occurred very rapidly. Within 10 minutes, specific Tm isoforms became polarised in their distribution. Other investigators have also found that changes in Tm and actin structure and composition occurs early in the development of cell structure. In a study by Temm-Grove et al, specific Tm isoform localisation occurred as soon as 15 minutes after being micro-injected into an epithelial cell (Temm-Grove et al., 1998, Cell Motility & the Cytoskeleton 40, 393-407). They found that Tm5 localised rapidly to the adhesion belt between adjacent cells. Other studies have examined expression levels with time. In fibroblasts, Tm 5NM isoform expression level increased 2-fold by 5 hours during the cell cycle (Percival et al., 2000, Cell Motility & the Cytoskeleton 47, 189-208). In cultured hepatocytes, F actin mass increased 20 fold within 30 minutes of cell adhesion to extra-cellular matrix (Mooney et al., 1995, Journal of Cell Science 108, 2311-2320). In developing neurones, Tm5 mRNA was found to localise to the axonal hillock thus forming an early marker of neuronal polarity (Hannan et al., 1995, Molecular & Cellular Neurosciences 6, 397-412). Thus we conclude that cells of various types are capable of rapidly altering their cytoskeletal structure either by increasing cytoskeletal protein expression or moving intact protein within the cell. These findings implicate Tm in the early processes of cell attachment and the development of polarisation.

Role of Tm5a and Tm5b on Regulating CFTR Function

Without wishing to be bound by theory, there are at least three possible mechanisms which may explain how Tm5a and Tm5b limit CFTR insertion into the apical membrane in response to cAMP stimulation. Firstly, that Tm5a and Tm5b may act as a physical barrier to vesicle movement towards the apical surface of the epithelial cell. When removed, vesicle movement would occur more freely and a subsequent increase in membrane inserted CFTR would be inevitable. Secondly, Tm5a and Tm5b may not act as a functional barrier to vesicle movement but may be an inhibitory control mechanism for the movement of vesicles along actin filaments. The movement of vesicles along actin filaments is an active process that requires the interaction of actin and myosin. Tm5a and Tm5b may inhibit this interaction. If this were the case, the presence of Tm5a and Tm5b in the apical region would be expected to inhibit the delivery of CFTR vesicles to the apical membrane. Conversely, depolarisation of Tm5a and Tm5b would be expected to increase the delivery of CFTR to the apical membrane. Finally, Tm5a and Tm5b associated microfilaments may be involved in the process of endocytic cycling of surface proteins. A study by Gottlieb et al found that microfilaments play a role in the endocytosis of proteins at the apical membrane of epithelial cells (Gottlieb et al., 1993, Journal of Cell Biology 120, 695-710). From their observations, they hypothesised that actin microfilaments form part of a mechanochemical motor that is involved in either moving microvillar membrane components towards the intervillar spaces or providing the force to convert membrane pits into endocytic vesicles. If the microfilaments involved in these processes contain Tm5a and Tm5b, then the removal of Tm5a and Tm5b would result in failure to endocytose proteins such as CFTR from the apical membrane.

Our finding that Tm5a and/or Tm5b regulate the insertion or retention of CFTR into the apical membrane contributes further to the growing body of evidence that Tm isoforms have different functions. There are over 40 Tm isoforms known to exist (Lees-Miller and Helfman, 1991, Bioessays 13, 429-437; Pittenger et al., 1994, Current Opinion in Cell Biology 6, 96-104) (Dufour et al., 1998, Journal of Biological Chemistry 273, 18547-18555). Supportive of the presence of differing functions is the knowledge that the various Tms confer different mechano-chemical properties to actin microfilaments. For example, the differing binding affinities of Tm isoforms for actin results in a differential effect on the stability of actin microfilaments (Pittenger et al., 1994, Current Opinion in Cell Biology 6, 96-104). Further evidence comes from work by Percival et al who found that Tm5NM confers greater cytochalasin D resistance to actin microfilaments (Percival et al., 2000, Cell Motility & the Cytoskeleton 47, 189-208). Others have found that specific Tm isoforms increase the rigidity of actin filaments (Kojima et al., 1994, Proceedings of the National Academy of Sciences of the United States of America 91, 12962-12966). Once inserted into the actin microfilament, Tms influence the interaction between actin and other actin binding proteins. For example, high molecular weight Tms are protective against the severing activity of the actin binding protein gelsolin (Ishikawa et al., 1989, Journal of Biological Chemistry 264, 7490-7497).

Our findings contribute to a growing body of evidence supporting a role for Tms in specific cellular functions. We conclude that Tm isoforms are segregated in gastrointestinal epithelial cells and are capable of regulating important cellular functions.

All documents referred to above by reference are incorporated in their entirety into this disclosure.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-43. (canceled)

44. A method of screening for a compound that regulates an activity of a cell surface protein, the method comprising analysing an activity or cellular location of tropomyosin, expression levels of tropomyosin, or binding of tropomyosin to one of its binding partners in the presence of a candidate compound, wherein altered tropomyosin activity or cellular location, altered expression levels of tropomyosin or an altered level of binding of tropomyosin to its binding partner in the presence of the compound indicates that the compound regulates the activity of a cell surface protein.

45. The method of claim 44 wherein altered cellular location of tropomyosin in the presence of the compound indicates that the compound increases the activity of a cell surface protein.

46. The method of claim 44 wherein reduced tropomyosin expression in the presence of the compound indicates that the compound increases the activity of a cell surface protein.

47. The method of claim 44 wherein a reduced level of binding of tropomyosin to its binding partner in the presence of the compound indicates that the compound increases the activity of a cell surface protein.

48. The method of claim 44 wherein the tropomyosin binding partner is selected from the group consisting of calponin, CEACAM1, endostatin, Enigma, Gelsolin, S100A2 and actin.

49. The method of claim 48 wherein the tropomyosin binding partner comprises sub-domain 2 of Gelsolin.

50. The method of claim 44 wherein the cell surface protein is selected from the group consisting of a transport protein, a channel, a receptor, a growth factor, an antigen, a signalling protein and a cell adhesion protein.

51. The method of claim 44 wherein the protein is a transport protein or a channel.

52. The method of claim 44 wherein the tropomyosin is a tropomyosin isoform comprising an amino acid sequence encoded by exon 1b of a TPM 1 gene (SEQ ID NO:11) or an amino acid sequence encoded by exon 1b of a TPM 3 gene (SEQ ID NO:12).

53. The method of claim 44 wherein the tropomyosin is a tropomyosin isoform TM5a or TM5b.

54. A method of screening for a therapeutic compound for treatment of cystic fibrosis, the method comprising analysing an activity or cellular location of tropomyosin, expression levels of tropomyosin or binding of tropomyosin to one of its binding partners in the presence of a candidate compound, wherein altered tropomyosin activity or cellular location, altered expression levels of tropomyosin or an altered level of binding of tropomyosin to its binding partner in the presence of the compound indicates that the compound is useful for treatment of cystic fibrosis.

55. The method of claim 54 wherein altered cellular location of tropomyosin in the presence of the compound indicates that the compound is useful for treatment of cystic fibrosis.

56. The method of claim 54 wherein reduced tropomyosin expression in the presence of the compound indicates that the compound is useful for treatment of cystic fibrosis.

57. The method of claim 54 wherein a reduced level of binding of tropomyosin to its binding partner in the presence of the compound indicates that the compound is useful for treatment of cystic fibrosis.

58. The method of claim 54 wherein the tropomyosin binding partner is selected from the group consisting of calponin, CEACAM1, endostatin, Enigma, Gelsolin, S100A2 and actin.

59. The method of claim 58 wherein the tropomyosin binding partner comprises sub-domain 2 of Gelsolin.

60. The method of claim 54 wherein the tropomyosin is a tropomyosin isoform comprising an amino acid sequence encoded by exon 1b of the TPM 1 gene (SEQ ID NO:11) or an amino acid sequence encoded by exon 1b of the TPM 3 gene (SEQ ID NO:12).

61. The method of claim 54 wherein the tropomyosin is a tropomyosin isoform TM5a or TM5b.

62. The method of claim 54 further comprising formulating the compound for administration to a human or a non-human animal.

63. A method for regulating insertion or retention of a protein in a cell surface membrane, the method comprising administering to a cell an agent that modulates tropomyosin expression, location or activity.

64. The method of claim 63 wherein the insertion or retention of the protein in the cell surface membrane is increased by administering a tropomyosin antagonist to the cell.

65. The method of claim 63 wherein the protein is selected from the group consisting of a transport protein, a channel, a receptor, a growth factor, an antigen, a signalling protein and a cell adhesion protein.

66. The method of claim 65 wherein the transport protein is cystic fibrosis transmembrane conductance regulator (CFTR).

67. The method of claim 63 wherein the tropomyosin is a tropomyosin isoform comprising an amino acid sequence encoded by exon 1b of the TPM 1 gene (SEQ ID NO:11) or an amino acid sequence encoded by exon 1b of the TPM 3 gene (SEQ ID NO:12).

68. The method of claim 63 wherein the tropomyosin is a tropomyosin isoform TM5a or TM5b.

69. The method of claim 64 wherein the tropomyosin antagonist is an antisense compound, a catalytic molecule or an RNAi molecule directed against tropomyosin-encoding mRNA.

70. The method of claim 64 wherein the tropomyosin antagonist is an antisense compound, a catalytic molecule or an RNAi molecule targeted specifically against exon 1b of a TPM 1 gene (SEQ ID NO:7) or exon 1b of a TPM 3 gene (SEQ ID NO:8).

71. The method of claim 64 wherein the tropomyosin antagonist is an antisense compound, a catalytic molecule or an RNAi molecule targeted to a sequence AGCTCGCTGGAGGCGGTG (SEQ ID NO:13).

72. A method for the treatment or prevention of cystic fibrosis in a subject, the method comprising administering to a subject an agent that modulates tropomyosin expression, location or activity.

73. The method of claim 72 wherein the tropomyosin is a tropomyosin isoform comprising an amino acid sequence encoded by exon 1b of the TPM 1 gene (SEQ ID NO:11) or an amino acid sequence encoded by exon 1b of the TPM 3 gene (SEQ ID NO:12).

74. The method of claim 72 wherein the tropomyosin is a tropomyosin isoform TM5a or TM5b.

75. The method of claim 72 wherein the agent that modulates tropomyosin expression, location or activity is an antisense compound, a catalytic molecule or an RNAi molecule directed against tropomyosin-encoding mRNA.

76. The method of claim 72 wherein the agent that modulates tropomyosin expression, location or activity is an antisense compound, a catalytic molecule or an RNAi molecule targeted specifically against exon 1b of s TPM 1 gene (SEQ ID NO:7) or exon 1b of a TPM 3 gene (SEQ ID NO:8).

77. The method of claim 72 wherein the agent that modulates tropomyosin expression, location or activity is an antisense compound comprising a sequence CACCGCCUCCAGCGAGCT (SEQ ID NO:14).


SEQUENCE LISTING (20 sequences)

SEQ ID NO: 1 - DNA, 1265 bp, Homo sapiens
ggcacgagggaggaatgcggtcgcccccttgggaaagtacatatctgggagaagcaggcggctccgcgctcgcactcccgctcctccgcccgaccgcgcgctcgccccgccgctcctgctgcagccccagggcccctcgccgccgccaccatggacgccatcaagaagaagatgcagatgctgaagctcgacaaggagaacgccttggatcgagctgagcaggcggaggccgacaagaaggcggcggaagacaggagcaagcagctggaagatgagctggtgtcactgcaaaagaaactcaagggcaccgaagatgaactggacaaatattctgaggctctcaaagatgcccaggagaagctggagctggcagagaaaaaggccaccgatgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggcaaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagacgagctgtacgctcagaaactgaagtacaaagccatcagcgaggagctggaccacgctctcaacgatatgacttccatgtaaacgttcatccactctgcctgcttacaccctgccctcatgctaatataagtttctttgcttcacttctcccaagactccctcgtcgagctggatgtcccacctctctgagctctgcatttgtctattctccagctgaccctggttctctctcttagcatcctgccttagagccaggcacacactgtgctttctattgtacagaagctcttcgtttcagtgtcaaataaacactgtgtaagctaaaaaaaaaaaaaaaaaaa

SEQ ID NO: 2 - DNA, 1044 bp, Homo sapiens
tgctgctctcctcccgctccgtcctcctcgcctgccaccggtgcacccagtccgctcacccagcccagtccgtccggtcctcaccgcctgccggccggcccaccccccaccgcaggccatggacgccatcaagaagaagatgcagatgctgaagctggacaaggagaacgccatcgaccgcgccgagcaggccgaagccgacaagaagcaagctgaggaccgctgcaagcagctggaggaggagcagcaggccctccagaagaagctgaaggggacagaggatgaggtggaaaagtattctgaatccgtgaaggaggcccaggagaaactggagcaggccgagaagaaggccactgatgctgaggcagatgtggcctccctgaaccgccgcattcagctggttgaggaggagctggaccgggcccaggagcgcctggctacagccctgcagaagctggaggaggccgagaaggcggctgatgagagcgagagaggaatgaaggtcatcgaaaaccgggccatgaaggatgaggagaagatggaactgcaggagatgcagctgaaggaggccaagcacatcgctgaggattcagaccgcaaatatgaagaggtggccaggaagctggtgatcctggaaggagagctggagcgctcggaggagagggctgaggtggccgagagccgagccagacagctggaggaggaacttcgaaccatggaccaggccctcaagtccctgatggcctcagaggaggagtattccaccaaagaagataaatatgaagaggagatcaaactgttggaggagaagctgaaggaggctgagacccgagcagagtttgccgagaggtctgtggcaaagttggagaaaaccatcgatgacctagaagagaccttggccagtgccaaggaggagaacgtcgagattcaccagaccttggaccagaccctgctggaactcaacaacctgtgagggccagccccacccccagccaggctatggttgccaccccaacccaataaaactgatgttactagcctctc

SEQ ID NO: 3 - DNA, 2089 bp, Homo sapiens
ggcacgaggggaggcaggaaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggctgaggctgaggtggcctccttgaaccgtaggatccagctggttgaagaagagctggaccgtgctcaggagcgcctggccactgccctgcaaaagctggaagaagctgaaaaagctgctgatgagagtgagagaggtatgaaggttattgaaaaccgggccttaaaagatgaagaaaagatggaactccaggaaatccaactcaaagaagctaagcacattgcagaagaggcagataggaagtatgaagaggtggctcgtaagttggtgatcattgaaggagacttggaacgcacagaggaacgagctgagctggcagagtcccgttgccgagagatggatgagcagattagactgatggaccagaacctgaagtgtctgagtgctgctgaagaaaagtactctcaaaaagaagataaatatgaggaagaaatcaagattcttactgataaactcaaggaggcagagacccgtgctgagtttgctgagagatcggtagccaagctggaaaagacaattgatgacctggaagataaactgaaatgcaccaaagaggagcacctctgtacacaaaggatgctggaccagaccctgcttgacctgaatgagatgtagaacgccccagtcccaccctgctgctgctcctccctctgacccagactccgcctgaggccagcctgcgggaagctgacctttaactgagggctgatctttaactggaaggctgctttctcctttcaccaccccctccttccctgtgtctttttcgccaaactgtctctgcctcttcccggagaatccagctgggctagaggctgagcacctttggaaacaacatttaagggaatgtgagcacaatgcataatgtctttaaaaagcatgttgtgatgtacacattttgtaattaccttttttgttgttttgtagcaaccatttgtaaaacattccaaataattccacagtcctgaagcagcaatcgaatccctttctcacttttggaaggtgacttttcaccttaatgcatattcccctctccatagaggagaggaaaaggtgtaggcctgccttaccgagagccaaacagagcccagggagactccgctgtgggaaacctcattgttctgtacaaagtactagctaaaccagaaaggtgattccaggaggagttagccaaacaacaacaaaaacaaaaaatgtgctgttcaagttttcagctttaagatatctttggataatgttatttctattttttatttttttcattagaagttaccaaattaagatggtaagacctctgagaccaaaattttgtcccatctctaccccctcacaactgcttacagaatggatcatgtcccccttatgttgaggtgaccacttaattgctttcctgcctccttgaaagaaagaaagaaagaagactgtgtttttgccactgatttagccatgtgaaactcatctcattacccttttctgggtttgaagctgctgtctctagaagtgccatctcaattgtgctttgtatcagtcagtgctggagaaatcttgaatagcttatgtacaaaactttttaaattttatattattttgaaactttgctttgggtttgtggcaccctggccaccccatctggctgtgacagcctctgcagtccgtgggctggcagtttgttgatcttttaagtttccttccctacccagtccccattttctggtaaggtttctaggaggtctgttaggtgtacatcctgcagcttattggcttaaaatgtactctccttttatgtggtctctttggggccgattgggagaaagagaaatcaatagtgcaactgttttgatactgaatattgacaagtgtctttttgaaataaagaaccagtccctccaaaaaaaaaaaaaaaaaa

SEQ ID NO: 4 - DNA, 2049 bp, Homo sapiens
gagcccagccgagcgtccgccgctgcccgtgcgcctctgcgctccgcgccatggccggcctcaactccctggaggcggtgaaacgcaagatccaggccctgcagcagcaggcggacgaggcggaagaccgcgcgcagggcctgcagcgggagctggacggcgagcgcgagcggcgcgagaaagctgaaggtgatgtggccgccctcaaccgacgcatccagctcgttgaggaggagttggacagggctcaggaacgactggccacggccctgcagaagctggaggaggcagaaaaagctgcagatgagagtgagagaggaatgaaggtgatagaaaaccgggccatgaaggatgaggagaagatggagattcaggagatgcagctcaaagaggccaagcacattgcggaagaggctgaccgcaaatacgaggaggtagctcgtaagctggtcatcctggagggtgagctggagagggcagaggagcgtgcggaggtgtctgaactaaaatgtggtgacctggaagaagaactcaagaatgttactaacaatctgaaatctctggaggctgcatctgaaaagtattctgaaaaggaggacaaatatgaagaagaaattaaacttctgtctgacaaactgaaagaggctgagacccgtgctgaatttgcagagagaacggttgcaaaactggaaaagacaattgatgacctggaagagaaacttgcccaggccaaagaagagaacgtgggcttacatcagacactggatcagacactaaacgaacttaactgtatataagcaaaacagaagagtcttgttccaacagaaactctggagctccgtgggtctttctcttctcttgtaagaagttccttttgttattgccatcttcgctttgctggaaatgtcaagcaaattatgaatacatgaccaaatattttgtatcggagaagctttgagcaccagttaaatctcattccttccctttttttttcaaatggcaccagctttttcagctctcttattttttccttaagtagcatttattcctaaggtaggcagggtatttcctagtaagcatactttcttaagacggaggccatttggttcctgggagaataggcagccccacactttgaagaatacagaccccagtatctagtcgtggatataattaaaacgctgaagaccataaccttttgggtcaactgttggtcaaactataggagagaccagggaccatcacatgggtagggattttccatccagagccaataaaaggactggtgggggccgggggtggctattgtgggaagtcataacccacagatagatcaacctaagaatcctggcccttctccactctccaccatgcaggacaaacatcttctcaagcagtcaacgtagaatgcttgggaaatagtcataattacccacatatagtaattaatagatggtaattaattgatccttgatgtgatgttcttttgcatatttccttcattctaaagttgttccctggccgggagcgtttgctttcgcctgtaatcccaacactttgggaggccaggacagatcacttgaggtcaggagttcgagaccagcccagccaacatggcgaaaccatgtctctactaaaaatacaaaaattatggtgacgcctgcctgtagtcccagctactcgggaggctgaggcaggaggatcgcttgaacccaggaagtggagactgcagtgagccgatatcgcaccacagcgctccagcctggtcgacagagtgagactccatctcaagaaaaaataaaaataaagttgttctctgaagagcaaatgtctcattccagtaatgacccactcagcaggaatatggtggagttcagtccaattcaggtcagccatatccaaaagaccacaagtcattactaagttgagcaaaagagtttttatctattagcagaaagggcctctctggcagcagagattaaaaactggcccaacttcatttccatacttcagggaacagcaaattgaggatttacttatctaggactt

SEQ ID NO: 5 - DNA, 1593 bp, Hono sapiens
ggcggaccggcgctgggcagccaggacagccgcggcagccgggtccgcagggcagcagccggcctctcccactgcagccctcccgcccgcctaccgtccggcgcgatggcggggagtagctcgctggaggcggtgcgcaggaagatccggagcctgcaggagcaggcggacgccgctgaggagcgcgcgggcaccctgcagcgcgagctggaccacgagaggaagctgagggagaccgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggccaagcacattgctgaagatgccgaccgcaaatatgaagaggtggcccgtaagctggtcatcattgagagcgacctggaacgtgcagaggagcgggctgagctctcagaaggcaaatgtgccgagcttgaagaagaattgaaaactgtgacgaacaacttgaagtcactggaggctcaggctgagaagtactcgcagaaggaagacagatatgaggaagagatcaaggtcctttccgacaagctgaaggaggctgagactcgggctgagtttgcggagaggtcagtaactaaattggagaaaagcattgatgacttagaagagaaagtggctcatgccaaagaagaaaaccttagtatgcatcagatgctggatcagactttactggagttaaacaacatgtgaaaacctccttagctgcgaccacattctttcgttttgttttgttttgtttttaaacacctgcttaccccttaaatgcaatttatttacttttaccactgtcacagaaacatccacaagataccagctaggtcagggggtggggaaaacacatacaaaaaggcaagcccatgtcagggcgatcctggttcaaatgtgccatttcccgggttgatgctgccacactttgtagagagtttagcaacacagtgtgcttagtcagcgtaggaatcctcactaaagcagaagaagttccattcaaagtgccaatgatagagtcaacaggaaggttaatgttggaaacacaatcaggtgtggattggtgctactttgaacaaaaggtccccctgtggtcttttgttcaacattgtacaatgtagaactctgtccaacactaatttattttgtcttgagttttactacaagatgagactatggatcccgcatgcctgaattcactaaagccaagggtctgtaagccacgctgctcttccgagacttccattcctttctgattggcacacgtgcagctcatgacaatctgtaggataacaatcagtgtggatttccactcttttcagtccttcatgttaaagatttagacaccacatacaactggtaaaggacgttttcttgagagttttaactatatgtaaacattgtataatgatatggaataaaatgcacattgtaggacattttcta

SEQ ID NO: 6 - DNA, 1593 bp, Homo sapiens
ggcggaccggcgctgggcagccaggacagccgcggcagccgggtccgcagggcagcagccggcctctcccactgcagccctcccgcccgcctaccgtccggcgcgatggcggggagtagctcgctggaggcggtgcgcaggaagatccggagcctgcaggagcaggcggacgccgctgaggagcgcgcgggcaccctgcagcgcgagctggaccacgagaggaagctgagggagaccgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggccaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagagaaagtggctcatgccaaagaagaaaaccttagtatgcatcagatgctggatcagactttactggagttaaacaacatgtgaaaacctccttagctgcgaccacattctttcgttttgttttgttttgtttttaaacacctgcttaccccttaaatgcaatttatttacttttaccactgtcacagaaacatccacaagataccagctaggtcagggggtggggaaaacacatacaaaaaggcaagcccatgtcagggcgatcctggttcaaatgtgccatttcccgggttgatgctgccacactttgtagagagtttagcaacacagtgtgcttagtcagcgtaggaatcctcactaaagcagaagaagttccattcaaagtgccaatgatagagtcaacaggaaggttaatgttggaaacacaatcaggtgtggattggtgctactttgaacaaaaggtccccctgtggtcttttgttcaacattgtacaatgtagaactctgtccaacactaatttattttgtcttgagttttactacaagatgagactatggatcccgcatgcctgaattcactaaagccaagggtctgtaagccacgctgctcttccgagacttccattcctttctgattggcacacgtgcagctcatgacaatctgtaggataacaatcagtgtggatttccactcttttcagtccttcatgttaaagatttagacaccacatacaactggtaaaggacgttttcttgagagttttaactatatgtaaacattgtataatgatatggaataaaatgcacattgtaggacattttcta

SEQ ID NO: 7 - DNA, 237 bp, Homo sapiens
ggcggaccggcgctgggcagccaggacagccgcggcagccgggtccgcagggcagcagccggcctctcccactgcagccctcccgcccgcctaccgtccggcgcgatggcggggagtagctcgctggaggcggtgcgcaggaagatccggagcctgcaggagcaggcggacgccgctgaggagcgcgcgggcaccctgcagcgcgagctggaccacgagaggaagctgagggagacc

SEQ ID NO: 8 - DNA, 203 bp, Homo sapiens
gggtgagaggaggctgcaacgccgagcgaggaggcaggaaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacag

SEQ ID NO: 9 - protein, 248 aa, Homo sapiens
MAGSSSLEAVRRKIRSLQEQADAAEERAGTLQRELDHERKLRETAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEEKVAHAKEENLSMHQMLDQTLLELNNM

SEQ ID NO: 10 - protein, 248 aa, Homo sapiens
MAGSSSLEAVRRKIRSLQEQADAAEERAGTLQRELDHERKLRETAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDRYEEEIKVLSDKLKEAETRAEFAERSVTKLEKSIDDLEEKVAHAKEENLSMHQMLDQTLLELNNM

SEQ ID NO: 11 - protein, 44 aa, Homo sapiens
MAGSSSLEAVRRKIRSLQEQADAAEERAGTLQRELDHERKLRET

SEQ ID NO: 12 - protein, 44 aa, Homo sapiens
MAGITTIEAVKRKIQVLQQQADDAEERAERLQREVEGERRAREQ

SEQ ID NO: 13 - DNA, 18 bp, Homp sapiens
agctcgctggaggcggtg

SEQ ID NO: 14 - DNA, 18 bp, Artificial sequence
  Antisense oligonucleotide
caccgccuccagcgagct

SEQ ID NO: 15 - DNA, 18 bp, Artificial sequence
  Oligonucleotide
gctccagccacgccgact

SEQ ID NO: 16 - RNA, 21 nt, Artificial sequence
  Oligonucleotide
gauccggagccugcaggaguu

SEQ ID NO: 17 - RNA, 21 nt, Artificial sequence
  Oligonucleotide
cuccugcaggcuccggaucuu

SEQ ID NO: 18 - protein, 9 aa, Artificial sequence
  Fragment of the protein encoded by exon 1b of human Tm5a and Tm5b
SLEAVRRKI

SEQ ID NO: 19 - protein, 9 aa, Artificial sequence
  Fragment of the protein encoded by exon 1b of human Tm5a and Tm5b
SLQEQADAA

SEQ ID NO: 20 - protein, 9 aa, Artificial sequence
  Fragment of the protein encoded by exon 1b of human Tm5a and Tm5b
RELDHERKL